(19) United States
(12) Patent Application Publication
(10) Pub. No.: US 2017/0080426 A1
(43) Pub. Date: Mar. 23, 2017

(54) DEVICE AND METHOD FOR TESTING COMPOUNDS ON LIVING CELLS

(71) Applicant: University of Limerick, Limerick (IE)

(72) Inventor: Mark Davies, Limerick (IE)

(21) Appl. No.: 15/310,613

(22) PCT Filed: May 14, 2015
(86) PCT No.: PCT/IB2015/001469

§ 371 Date: Nov. 11, 2016

Related U.S. Application Data
(60) Provisional application No. 62/115,872, filed on Feb. 13, 2015.
(60) Provisional application No. 62/115,877, filed on Feb. 13, 2015.
(60) Provisional application No. 61/993,119, filed on May 14, 2014.

(51) Int. Cl. B01L 3/00; B01F 13/00; C12M 3/06; G01N 33/50; C12Q 1/18

(52) U.S. Cl. CPC B01L 3/502784; G01N 33/5011; G01N 33/5044; C12Q 1/18; C12M 23/16; B01F 13/0071; B01F 13/0083; B01F 13/0076; B01L 2200/0673; B01L 2300/0627; B01L 2300/18; B01L 2400/0688; B01L 2200/027; B01L 2400/0415; B01L 2300/16; B01L 2200/12; B01L 2400/065

(57) ABSTRACT

The invention provides mechanical devices that can be used to combine reagents and discover their effects on living cells. A device of the invention holds liquids in open-ended channels and transfers liquids from one channel to another by bringing a second channel into proximity and alignment with an open end of a first channel. Channels of the device can include fluid partitions (e.g., water-oil-water emulsions) that include living cells. The device includes a mechanical system the operation of which causes a receiving channel to pass among supply channels, aligning with each in turn, to pick up chemicals from those channels and make new combinations within the receiving channel. The device then presents those new chemical combinations to the living cells within their respective partitions, thereby allowing for the determination of the effects of those combinations on living cells.

### RELATED APPLICATIONS

This application claims the benefit of and priority to U.S. provisional application No. 61/993,119 filed May 14, 2014; U.S. provisional application No. 62/115,872 filed Feb. 13, 2015; and U.S. provisional No. 62/115,877 filed Feb. 13, 2015, each of which is incorporated by reference.

### FIELD OF THE INVENTION

The invention generally relates to methods of disease modeling in living cells in a microfluidic device.

### BACKGROUND

Despite the wide variety of drugs currently on the market, people still suffer from diseases for which there is no satisfactory drug. For example, there is no cure for Type 2 Diabetes, a chronic disease afflicting millions of Americans. Instead of a cure, an afflicted person must submit to insulin therapy which requires daily monitoring of blood sugar levels and daily insulin injections. Even with insulin therapy, a person with Type 2 Diabetes faces the potential damage to nerves and small blood vessels of the eyes, kidneys, and heart. Damage to these tissues ultimately results in heart attack or stroke.

As another example, hospital-acquired staph infections are particularly troublesome. Many strains of staphylococcus encountered in hospitals are resistant to known antibiotics. Patient care would be greatly improved if researchers were to discover a drug that could knock out antibiotic resistant bacteria.

One approach to developing new drugs involves combining existing drugs into new compounds and screening those compounds for beneficial and unexpected effects. However, testing millions of possible drug combinations on living cells is costly and labor intensive. To make millions of combinations of drugs and test each combination on living cells requires very expensive liquid-handling robots or thousands of hours of labor. Furthermore, the fragile nature of cells further complicates the process as cells are incompatible with high through-put systems. For example, current high throughput systems use pumps and electrodes to control fluid flow, but the pumps and electrodes kill the cells before the experiment is concluded.

### SUMMARY

The invention provides mechanical devices that can be used to combine reagents and discover their effects on living cells in high-throughput screenings, thus allowing tens of millions of novel compounds to be rapidly screened to discover a compound with lifesaving potential. Devices of the invention use gravity and surface tension to combine liquids and control flow, thus avoiding pumps and electrodes and providing an environment suitable to living cells. Open-ended channels retain liquid by surface tension and liquids flows from one channel to another only when those channels are aligned and in proximity. Liquids containing agents, such as drugs or chemicals, are held in one or more individual source channels. The device includes a mechanical system that controls the operation of channels relative to one another. A receiving channel passes among the source channels, aligning with each in turn, to pick up liquids from those channels. The receiving channel can combine one, two, three, four, or any number of drugs in a fluid partition. Living cells can be introduced into the fluid partitions by operation of the device. The partitions can be, for example, water-in-oil-in-water emulsions, allowing the cells to be isolated within the partitions but also allowing nutrients and waste to diffuse into and out of the partitions. By exposing living cells to millions of compounds, each representing a new combination of drugs, devices of the invention can be used to assay for the effects of those compounds on the cells. Since the invention provides devices for creating and analyzing millions of combinations of drugs, devices of the invention may thus be used to discover unexpected or synergistic effects for new combinations of known drugs and to possibly discover a valuable antibiotic or a treatment for a disease.

In certain aspects, the invention provides a method for analyzing cells in partitions. The method includes holding a living cell in a fluid partition in a first open-ended channel and introducing a liquid that includes an agent into the partition by aligning at least a portion of the first open ended channel with a second open ended channel from which the fluid is provided. The contents of the fluid partition are analyzed, e.g., to determine an effect of the agent on the living cell. Preferably, aligning comprises contacting a liquid in the first channel with a liquid in the second channel. Any suitable cell can be held in the fluid partition such as a stem cell (e.g., a pluripotent stem cell modelling a disease state). The cell may be infected with a contagious disease or it may be exposed to a virus, pathogen, or bacteria. A concentration gradient may be created to remove waste from the fluid partition. In some embodiments, the agent in the liquid includes at least one drug molecule.

Aspects of the invention provide a method for analyzing cells in partitions. The method includes containing at least one cell in a first fluid compartment of a fluid partition in a first open ended channel—wherein the fluid partition comprises at least two fluid compartments—and introducing a fluid into the partition by aligning the first open ended channel with a second open ended channel from which the fluid is provided. The contents of the partition are analyzed. The first fluid compartment may contain a first cell type and a second fluid compartment may contain a second cell type. In some embodiments, the first fluid compartment contains a stem cell and a second fluid compartment contains an agent (e.g., a virus, a drug molecule, a bacterium, or a protein). The stem cell may be analyzed after incubation with the agent.

In some aspects, the invention provides a device for analyzing cells in partitions. The device includes a first open-ended channel configured to hold a living cell in a fluid partition and a second open-ended channel operably coupled to a control mechanism. Operation of the control mechanism aligns at least a portion of the first open-ended channel with the second channel, thereby introducing a liquid comprising an agent into the fluid partition. The device can be used to analyze the effects of the agent on the living cell. Preferably, aligning comprises contacting a liquid in the first channel with a liquid in the second channel. Any suitable cell can be held in the fluid partition such as a stem cell (e.g., a pluripotent stem cell (PSC) modelling a disease state). The cell may be infected with a contagious disease or it may be exposed to a virus, pathogen, or bacteria. A concentration gradient may be created to remove waste from the fluid partition. In some embodiments, the agent in the liquid includes at least one drug molecule.

Using method or devices of the invention, cells are isolated in fluid partitions, such as liposomes, micelles, emulsions, or droplets. An agent, such as a small molecule, a drug compound, a protein, etc., is introduced into the fluid partition. The cell, or the fluid within the partition, is analyzed for changes in cellular functions. By using the open-ended channel systems of the invention, cellular based assays are accomplished at a high-throughput level. Thus, various combinations of drugs, molecules, or agents can be tested against a wide number of cells to discover a possible cure to some of humanities most debilitating diseases, including Type 2 Diabetes.

Methods and devices of the invention use open-ended channels. The open-ended channels are oriented and configured so that fluid only flows between two open-ended channels when they are aligned. When the open-ended channels are not aligned, flow does not occur. Thus, fluids containing cells are manipulated and controlled in a gravity-based system that preserves cell integrity. By performing the methods of the invention in open ended channels, millions of cells are investigated and analyzed in a high throughput fashion.

Methods and devices of the invention may be used to analyze any suitable cell type. In some embodiments, the cells are stem cells. The stem cells or pluripotent stem cells can be induced to model a disease state, such as cancer, diabetes, Huntington's disease, rheumatoid arthritis, etc. In some embodiments, a disease-state cell is used. For example, beta-cells from a Type 2 Diabetes patient may be investigated or analyzed. In some embodiments, the stem cells may be infected with a contagious disease, such as a virus or a pathogen.

The methods of the invention may use a wide variety of cells, whether native cells, or induced disease state cells. In certain embodiments, the cells are exposed to an agent. The agent may be a drug compound, a combination of drug compounds, a protein, etc. The agent may be introduced into the partition containing the cell. After incubation for a period of time, the cell or the fluid around the cell may be analyzed to discern alterations in cellular function.

In some embodiments of the invention, a cell is contained within a fluid partition and the fluid partition contains multiple compartments. The fluid partition may contain two aqueous compartments, wherein at least one compartment contains a cell. An agent, virus, or pathogen may be found in the other compartment. A cell may be contained in each aqueous fluid compartment with an agent introduced into each compartment. After a period of incubation, alterations in cellular function or cellular products are investigated and analyzed.

Genetically modified cells may be used to model a disease. Various cell types, e.g. heart, lung, kidney, brain, etc., may be genetically modified or infected to model a disease. The modified or infected cells may be screened against a multitude of compounds, molecules, or agents to comprehensively investigate toxicity and efficacy. Thus, using the systems and methods of the invention, a wide variety of cell types, or cell models can be combined with various combinations of drugs.

### BRIEF DESCRIPTION OF THE DRAWINGS

FIGS. 1A-1B show an embodiment of a system of the invention.

FIGS. 2A-2C depict alternate configurations of the channels.

FIGS. 3A-3C depict a multi-channel system.

FIGS. 4A-4E depict a multi-channel system.

FIGS. 5A-5B depict a multi-compartment emulsion of the invention.

FIG. 6 depicts a microfluidic channel.

FIG. 7 depicts the results of off-patent drugs and drug combinations of MCF-7 cells.

FIG. 8 depicts a dose response curve of the effect of antibiotics on E. coli cells.

FIG. 9 depicts a dose response curve of the effects of antibiotics on E. coli cells.

FIG. 10 depicts a dose response curve for the effect of drugs on MCF-7 cells.

FIG. 11 depicts PSC drug treatments in droplets.

FIG. 12 depicts PSC in droplets.

FIG. 13 depicts cell growth and viability in droplets.

FIG. 14 depicts a schematic of the channels and fluid flow.

FIG. 15 is a graph of dispensing time versus droplet volume.

FIGS. 16A and 16B depict substrates on a mechanical subsystem.

### DETAILED DESCRIPTION

The invention provides methods and devices for analyzing cells in partitions. A device of the invention holds liquids in open-ended channels and transfers liquids from one channel to another by bringing a second channel into proximity and alignment with an open end of a first channel. Channels of the device can include fluid partitions (e.g., water-oil-water emulsions) that include living cells. The device includes a mechanical system the operation of which causes a receiving channel to pass among supply channels, aligning with each in turn, to pick up chemicals from those channels and make new combinations within the receiving channel. The device then presents those new chemical combinations to the living cells within their respective partitions. The contents of the fluid partition, including the cell, are analyzed.

Methods of the invention relate to forming multi-liquid partitions within a partition, where the liquid partitions contain at least one cell. The liquid partitions may contain the same type of cell, or differing cell types. Cells may be altered to express a disease, or may be transfected with a virus, bacteria, element, molecule, etc. Once infected, the cells may be exposed to a molecule, compound, or other agent. Multiple partitions may be processed and manipulated in sliding microfluidic channels. In some embodiments, the contents of the partitions or the cells are analyzed.

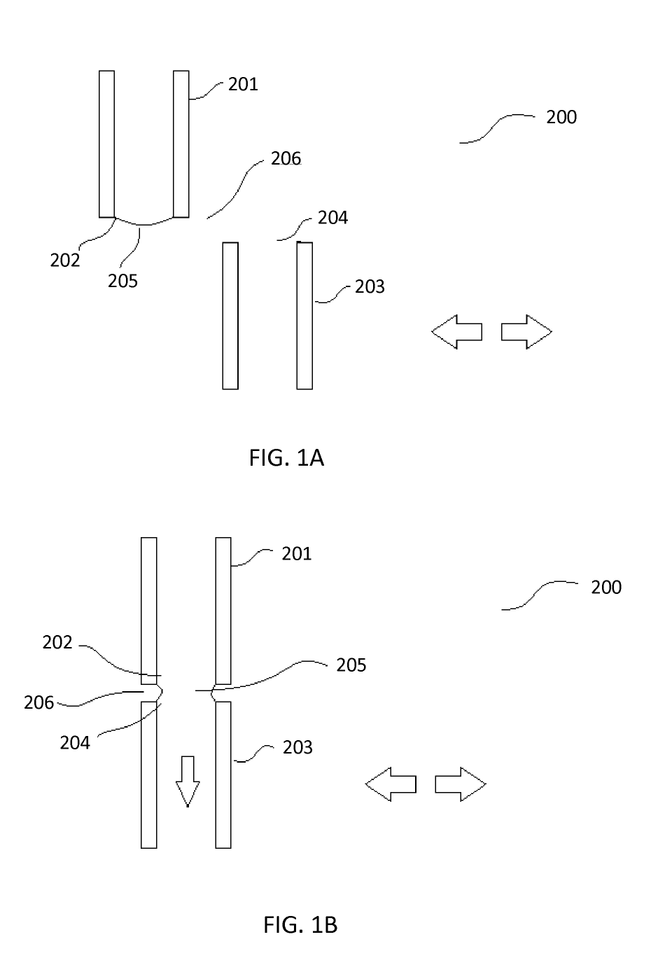

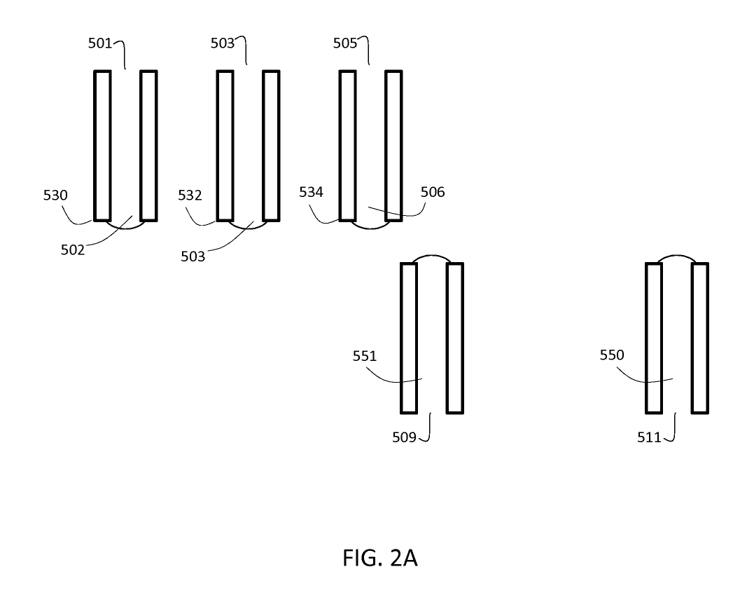

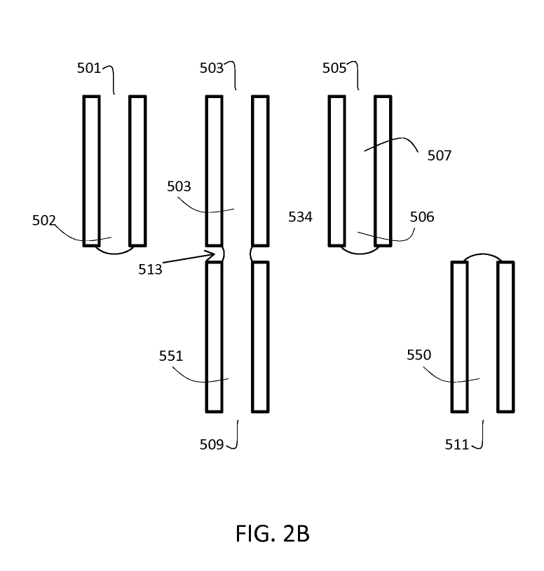

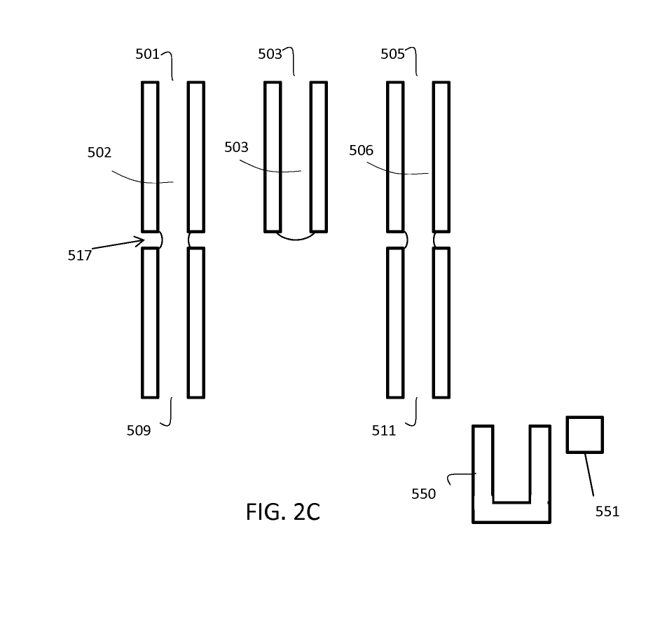

Methods of the invention are performed using open-ended channels. FIGS. 1A-1B show an exemplary embodiment of a microfluidic system 200 in which the methods of the invention are performed. FIGS. 1A-1B are described in the context of two channels for the sake of simplicity. However, the skilled artisan will recognize that the invention is not limited to two channels, and the invention encompasses systems designed with any number of channels, as will be described in additional embodiments below. Microfluidic system 200 includes a first channel 201 having an open end 202, and a second channel 203 having an open end 204. The first and second channels are slidable relative to each other such that when the open end 202 of the first channel 201 and the open end 204 of the second channel 203 are aligned with each other, fluid 205 flows from the first channel 201 into the second channel 203 (in FIG. 1B, flow is shown by a large downward pointing arrow within channel 203). When the first channel 201 and the second channel 203 are not aligned, fluid 205 does not flow within the first channel 201 and the second channel 203 (FIG. 2A).

Alignment of channels can include complete or partial alignment. In complete alignment, the center axes of two microfluidic channels are aligned. In partial alignment, the center axes are not aligned, however, there is partial overlap of the first and second channels such that the distance between the center axes is sufficiently small so that flow between the two microfluidic channels occurs. In complete misalignment, there is no overlap between the channels and the distance between the center axes is sufficiently great so that flow between the two microfluidic channels does not occur. In the present invention, alignment is meant to encompass both complete and partial alignment. The device of the invention flows fluid between two microfluidic channels even in the cases of partial alignment.

The channels may slide in any direction relative to each other, e.g., horizontally, vertically, diagonally, etc. In certain embodiments, the first channel 201 and the second channel 203 are horizontally slideable relative to each other as shown in FIGS. 1A-1B (horizontal arrows). FIGS. 1A-1B show second channel 203 being slideable relative to first channel 201, which remains stationary. However the invention is not limited to such a configuration. In other embodiments, it may be first channel 201 that is slidable relative to second channel 203, which remains stationary. In another embodiment, both the first channel 201 and the second channel 203 are slidable, that is, neither channel remains stationary and both channels are movable.

In certain embodiments, the open end 202 of the first channel 201 and the open end 204 of the second channel 203 are exposed to atmospheric pressure. In such embodiments, the first channel 201 and second channel 203 may be arranged in relation to each other such that an air gap 206 exists between the channels. As shown in FIGS. 1A-1B, when the open end 202 of the first channel 201 and the open end 204 of the second channel 203 are aligned with each other, fluid 205 from the first channel 201 bridges the air gap 206 and enters the second channel 203.

In an aspect of the invention, the air gap may comprise any known gas, at any suitable temperature and pressure. The air gap may be at atmospheric pressure and comprise of air. However, the air gap is not limited to atmospheric pressure or air. In some embodiments, the devices of the present invention may be completely or partially enclosed within a chamber and the chamber may be filled with a gas other than air. The pressure can be above or below atmospheric pressure and the temperature can be at, above, or below room temperature, which is about 37 degrees Celsius. In particular embodiments, gravitational force is used to produce and control flow within the system. As shown in FIGS. 1A-1B, the first channel 201 and the second channel 203 are arranged (e.g., arranged vertically) such that gravity causes flow of fluid 205 within the first channel 201 and second channel 203 when the open end 202 of the first channel 201 and the open end 204 of the second channel 203 are aligned with each other.

The first channel 201 and second channel 203 may be configured such that when they are not aligned, fluid 205 does not flow within the first channel 201 and/or second channel 203. That can be achieved in numerous different ways, such as by adjusting length of the channels, internal diameter of the channels, viscosity of the fluid(s) within the channels, surface tension of the fluid(s) within the channels, and/or density of the fluid(s) within the channels.

Methods of the invention are performed in the open ended, slidable channels shown in FIGS. 2A and 2B. In some embodiments, the fluid 205 contains partitions. In some embodiments, the channel 203 contains a fluid (not shown) and when aligned with channel 201, fluid 205 flows into channel 203 and then merges with a partition in channel 203.

It should be appreciated that methods of the invention may be carried out in multi-channel systems. There is no limit to the number of channels that can be included in systems of the invention, nor is there any limitation on the configuration of the channels. FIGS. 2A-2C depict a multi-channel system configured to allow microfluidic channels to slide or move relative to each other to alter alignment of the channels. FIG. 2A shows multiple microfluidic channels 501, 503, and 505 which are open at ends 530, 532, and 534. Microfluidic channels 501, 503, and 3505 each may contain a fluid, for example microfluidic channel 501 contains fluid 502. Fluids 502, 503, and 506 may be the same or different. Each fluid 502, 503, and 506 is retained in the microfluidic channels due to channel geometry and by forces such as surface tension. As discussed above, an aspect of the invention is that fluid does not flow from the microfluidic channel unless aligned with another microfluidic channel. Additionally, microfluidic channels 501, 503, and 505 may be slidable or moveable together or independent of one another. FIG. 2A also depicts microfluidic channels 509 and 511 which are open ended at 540 and 541. Microfluidic channels 509 and 511 are shown in FIG. 2A to contain fluids 550 and 551. However, microfluidic channels 509 and 511 are not required to contain fluids and may not contain fluids. Microfluidic channels 509 and 511 may be moved independent of one another or may be moved together. As shown in FIG. 2A, microfluidic channels 509 and 511 are positioned to be disengaged from microfluidic channels 501, 503, and 505. In this positioning of the microfluidic channels, microfluidic channels 501, 503, and 505 are prevented from flowing fluid due to the physical properties of the microfluidic channel and the fluid, e.g. surface tension.

Microfluidic channels 509 and 511 may be slid or moved to align with any of the microfluidic channels 501, 503, or 505. FIG. 2B depicts microfluidic channels 509 and 511 having been moved or slid relative to microfluidic channels 501, 503, or 505. As shown in FIG. 2B, microfluidic channel 509 has been slid to engage at least one of microfluidic channels 501, 503, and 505. Moving or sliding of microfluidic channel 509 or 511 may involve movement in any plane or direction. In FIG. 2B, microfluidic channel 503 is aligned with microfluidic channel 509. The alignment may cause an air gap 513. Also, as discussed above, it is not necessary for the microfluidic channels to be aligned so that the microfluidic channels are flush. Rather, an air gap 513 may be present between the two microfluidic channels. The arrangement of microfluidic channels 503 and 509 is such when the channels are aligned, fluid bridges the air gap 513 and flows from microfluidic channel 503 into microfluidic channel 509. In this positioning, microfluidic channel 509 receives fluid 503 from microfluidic channel 503.

The microfluidic channels of the invention may be slid or moved in several iterations. For example, as shown in FIG. 2C, microfluidic channel 509 has been slid to align with microfluidic channel 501. As discussed previously, microfluidic channel 509 was aligned with microfluidic channel 503 and received fluid 503. Microfluidic channel 509 now contains fluid 503 and fluid 502. In this embodiment, fluids are mixed from two different microfluidic channels. Additionally, as shown in FIG. 2C, microfluidic channel 511 is aligned with microfluidic channel 505. It should be appreciated that microfluidic channels 509 and 511 could be slid or moved at the same time, or independently of each other, depending on the configuration of microfluidic channels 509 and 511 and their respective substrates.

It should be appreciated that the multichannel systems of the invention may include numerous channels aligned in various planes of space. For example, FIGS. 2A-2C serve to illustrate how two levels of microfluidic channels can align to direct the flow of partitions within a microfluidic system. It should be appreciated that numerous levels of microfluidic channels may include a multichannel system, as discussed below.

The microfluidic device of the invention may be utilized to flow partitions within microfluidic channels. As discussed in detail above in regards to FIGS. 2A-2C, the microfluidic channels of the present invention can be aligned to transfer liquids from one to another. In some embodiments, the microfluidic system may be used to flow partitions within and between microfluidic channels. The partitions may be provided within a carrier fluid, such as an oil. In some embodiments, a fluid (fluid 502, see FIGS. 2A-2C) within a channel (channel 501) contains at least one partition. Aligning channel 509 with channel 502 allows for at least one partition to flow into channel 509. The partition can be a liposome, a droplet, etc. The partition can contain a cell, such as a stem cell or a pluripotent stem cell. Channel 509 can then align with channel 503 so that liquid within channel 503 flows into channel 509. The liquid in channel 509 merges or coalescences with the partition (discussed below). The liquid can contain any suitable agent such as a virus, pathogen, bacteria, drug, therapeutic agent, etc. The partition may be flowed into a container (see container 550 in FIG. 2C) and detected or analyzed using a detector 551.

It should be appreciated that the microfluidic systems may be coupled with additional devices to carry out the methods of the invention. For example, a detector may be included with or within devices of the invention. The detector can be optical or electrical detectors or combinations thereof. Examples of suitable detection apparatuses include optical waveguides, microscopes, diodes, light stimulating devices, (e.g., lasers), photo multiplier tubes, and processors (e.g., computers and software), and combinations thereof, which cooperate to detect a signal representative of a characteristic, marker, or reporter, and to determine and direct the measurement or the sorting action at a sorting module.

In some embodiments, a portion of the multichannel system comprising one or more microfluidic channels is in thermal contact with a thermal regulator. The thermal regulator can be any device that regulates temperature. This includes, for example, resistive wires that heat up when a voltage is applied, resistive heaters, fans for sending hot or cold air toward the isolated portion, Peltier devices, infrared (IR) heat sources such as projection bulbs, circulating liquids or gases in a contained device, and microwave heating.

A thermal regulator controls the temperature of liquids or partitions within a device of the invention, e.g., for incubation or control of chemical reactions. The ability of the thermal regulator to be programmed for different temperatures and incubation times, together with other aspects of the invention to control the introduction of samples, reactants and other reagents into the microfluidic channels provides the ability to control reaction times, temperatures, and reaction conditions within the microfluidic channels. For example, a portion of the multichannel system is in contact with a heat spreader of the thermal regulator. There may be an air gap between the portion of the microfluidic device and the heating element of the thermal regulator. The microfluidic device can be secured to the thermal regulator by one or more bolts, screws, pins, clips, brackets, or other such securing devices.

In certain embodiments of the present invention, the fluid within the microfluidic channel contains partitions. As discussed above, partitions may be composed or various fluids and various components. As discussed previously, the microfluidic channels of the invention can be slid to align in order to select the path of the partitions within the microfluidic system. As the partitions flow through the channels, flow can be stopped within a microfluidic channel by misalignment with another microfluidic channel. Flow may resume once the microfluidic channel is aligned with another microfluidic channel such that flow occurs therebetween.

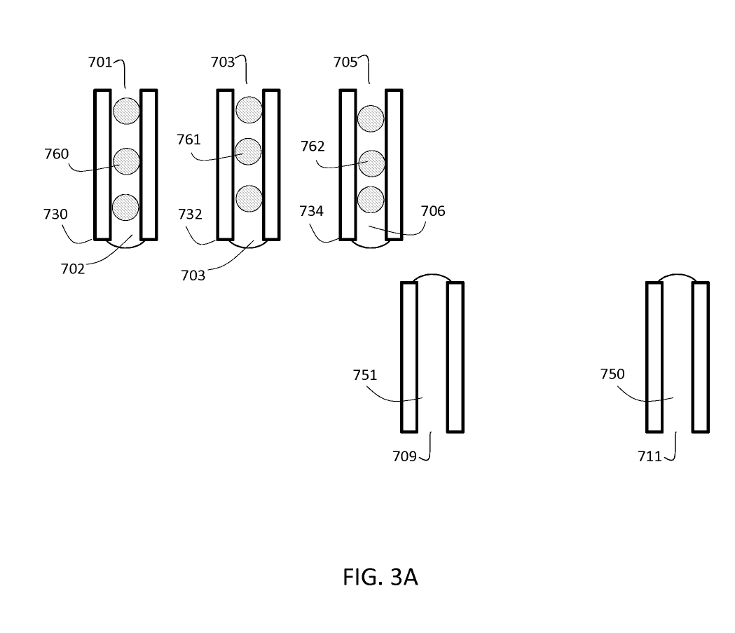

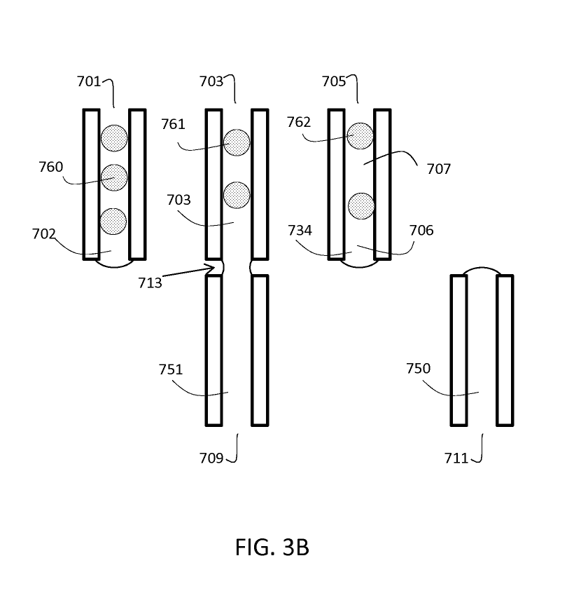

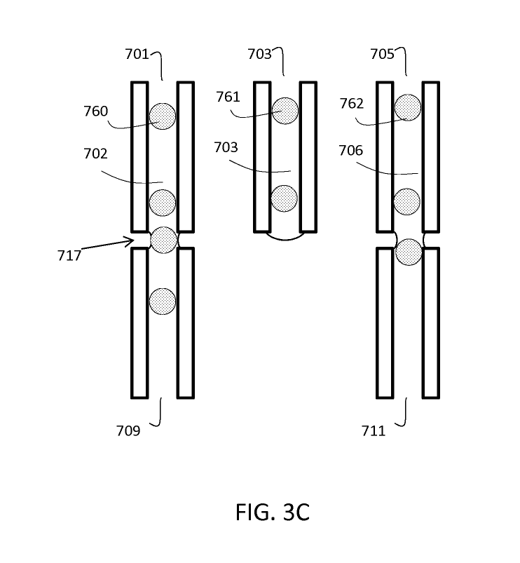

Devices and methods of the invention may use a multiple channel system. FIGS. 3A-3C depict a multi-channel system configured to allow microfluidic channels to slide or move relative to one another to alter alignment of the channels. FIG. 3A shows multiple microfluidic channels 701, 703, and 705 which are open at ends 730, 732, and 734. Microfluidic channels 701, 703, and 705 each may contain a liquid. For example, microfluidic channel 701 contains liquid 702. Liquids 702, 703, and 706 may be composed of the same components or may be composed of differing components. Each liquid 702, 703, and 706 is retained in the microfluidic channel by forces such as surface tension (without being bound by an mechanism, the force holding a liquid in an open-ended channel regardless of the presence of gravity may include contributions from surface tension, “wicking” or capillary forces, electrostatic forces, others, or combinations thereof). Liquid does not flow from the microfluidic channel unless aligned with another microfluidic channel. Each microfluidic channel 701, 703, and 705 may contain one or any number of partitions 760, 761, and 762. The partitions may be composed of various fluids and components. Additionally, the partitions 701, 703, and 705 may each include the same or different materials and components. Additionally, microfluidic channels 701, 703, and 705 may be slidable or moveable together or independent of one another. FIG. 3A also depicts microfluidic channels 709 and 711 which are open ended at 740 and 741. It should be appreciated that microfluidic channels 709 and 711 are shown in FIG. 3A to contain fluids 750 and 751. However, it should also be appreciated that microfluidic channels 709 and 711 may not contain liquids. Microfluidic channels 709 and 711 may be moved independent of one another or may be moved together. It is an aspect of the invention that microfluidic channels may be arranged in any configuration and manner. As shown in FIG. 3A, microfluidic channels 709 and 711 are positioned to be disengaged from microfluidic channels 701, 703, and 705. In this positioning of the microfluidic channels, microfluidic channels 701, 703, and 705 are prevented from flowing liquid due to the physical properties of the microfluidic channel and the immiscible fluid, e.g. surface tension, as discussed above.

Microfluidic channels 709 and 711 may be slid or moved to align with any of the microfluidic channels 701, 703, or 705. FIG. 3B depicts microfluidic channels 709 and 711 that has been moved or slid relative to microfluidic channels 701, 703, or 705. As shown in FIG. 3B, microfluidic channel 709 has been slid to engage at least one of microfluidic channels 701, 703, and 705. It should be appreciated that the moving or sliding of microfluidic channel 709 or 711 may involve movement in any plane or direction. In FIG. 3B, microfluidic channel 703 is aligned with microfluidic channel 709. The alignment may cause an air gap 713. Also, as discussed above, it is not necessary for the microfluidic channels to be aligned so that the microfluidic channels are flush. Rather, an air gap 713 may be present between the two microfluidic channels. The alignment of microfluidic channels 703 and 709 forms an air gap at 713. The formation of the air gap 713 results in a portion of fluid spanning air gap 713 and allows for fluid 703 and partitions 761 to flow from microfluidic channel 703 into microfluidic channel 709. In this positioning, microfluidic channel 709 receives fluid 703 and partitions 761 from microfluidic channel 703.

The microfluidic channels of the invention may be slid or move in several iterations. For example, as shown in FIG. 3C, microfluidic channel 709 has been slid to align with microfluidic channel 701. As discussed previously, microfluidic channel 709 was aligned with microfluidic channel 703 and received fluid 703 and partitions 761. Microfluidic channel 709 now contains liquid 703 and liquid 702 and partitions 761 and 760. In this embodiment, the components of the two different microfluidic channels are mixed. Additionally, as shown in FIG. 3C, microfluidic channel 711 is aligned with microfluidic channel 305. Microfluidic channels 709 and 711 can be slid or moved at the same time, or independently of each other, depending on the configuration of microfluidic channels 709 and 711 and their respective substrates.

Multichannel systems of the invention may include numerous channels aligned in various planes of space. For example, FIGS. 3A-3C serve to illustrate how two levels of microfluidic channels can align to direct the flow of partitions within a microfluidic system. It should be appreciated that different system architectures within the scope of the invention. It should be appreciated that the channels may be slid manually, by a gear, by a robotic stage, by a motor, etc.

Systems of the invention can also be used for partition merging or coalescing. The fluidic partitions may be of unequal size in certain cases. In certain cases, one or more series of partitions may each consist essentially of a substantially uniform number of entities of a species therein (i.e., molecules, cells, particles, etc.). The fluidic partitions may be coalesced to start a reaction, and/or to stop a reaction, in some cases. For instance, a reaction may be initiated when a species in a first partition contacts a species in a second partition after the partitions coalesce, or a first partition may contain an ongoing reaction and a second partition may contain a species that inhibits the reaction. Embodiments of the invention are directed to alignment of microfluidic channels to promote the coalescence of fluidic partitions.

Partitions may coalesce upon application of an electric field. The applied electric field may induce a charge, or at least a partial charge, on a fluidic partition surrounded by an immiscible fluid. Upon the application of an electric field, for example by producing a voltage across electrodes using a voltage source, partitions are induced to assume opposite charges or electric dipoles on the surfaces closest to each other, causing the partitions to coalesce.

In another aspect of the invention, two partitions may fuse by creating a liquid bridge between them, which may occur due to the charge-charge interactions or to reduce surface tensions. The creation of the liquid bridge between the two partitions allows the two partitions to exchange material or coalesce into one partition. Thus, in some embodiments, the invention provides for the coalescence of two separate partitions into one coalesced partition in systems where such coalescence ordinarily is unable to occur, e.g., due to size or surface tension.

Devices of the invention can also be used for partition merging or coalescing. The fluidic partitions may be of unequal size in certain cases. In certain cases, one or more series of partitions may each consist essentially of a substantially uniform number of entities of a species therein (i.e., molecules, cells, particles, etc.). The fluidic partitions may be coalesced to start a reaction, and/or to stop a reaction, in some cases. For instance, a reaction may be initiated when a species in a first partition contacts a species in a second partition after the partitions coalesce, or a first partition may contain an ongoing reaction and a second partition may contain a species that inhibits the reaction. Embodiments of the invention are directed to alignment of microfluidic channels to promote the coalescence of fluidic partitions.

In some embodiments, partitions are merged without the use of electrodes or electric fields. Coalescing of partitions without the application of an electric field is described by Xu et al., “Droplet Coalescence in Microfluidic Systems,” Micro and Nanosystems, 2011, 3, 131-136, which is incorporated by reference. The method described by Xu et al. merges droplets by allowing two droplets to have close contact with each other, causing the liquids of the two droplets to form a thin bridge between the two partitions. The methods described by Xu et al. are passive, where an external energy source is not needed. Passive merging can also be accomplished by channel geometry. See for example Gu et al., Int J Mol Sci. 2011; 12(4): 2572-2597, incorporated by reference. For example, partition merging may be accomplished using a widening channel follow by a narrower channel. In this geometry the partition velocity decreases in the widening channel, after which it increases again upon entry in the narrow channel. Partitions in close proximity merge with one another.

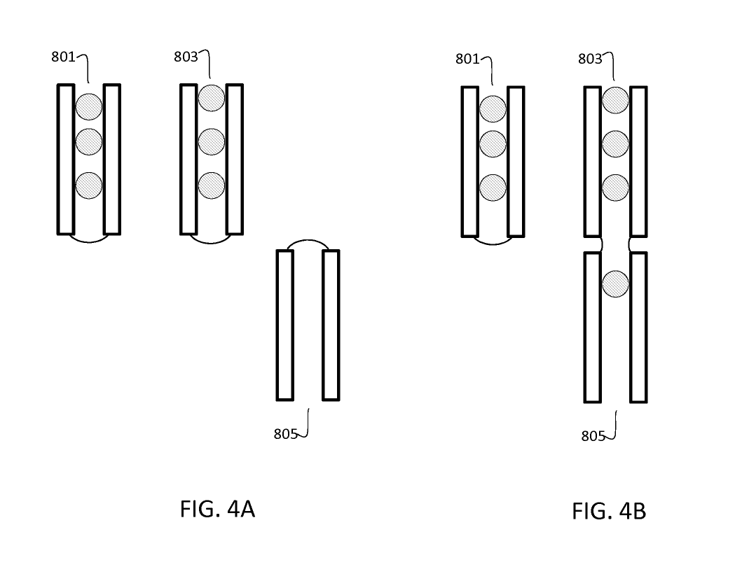

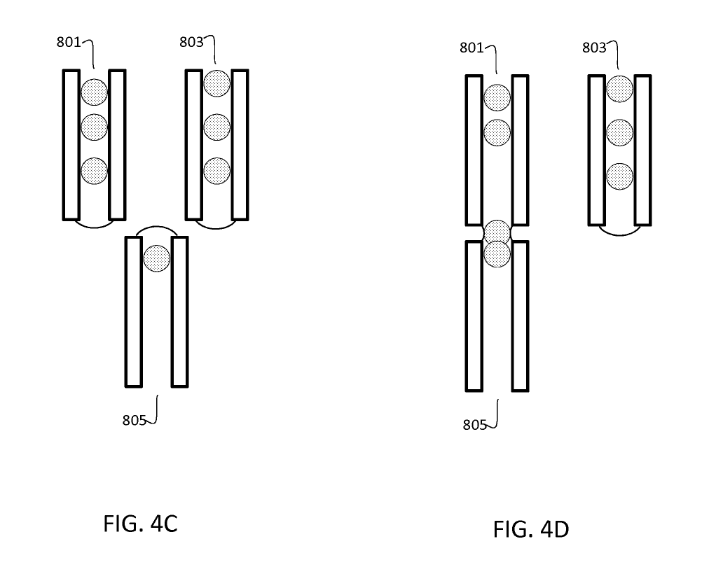

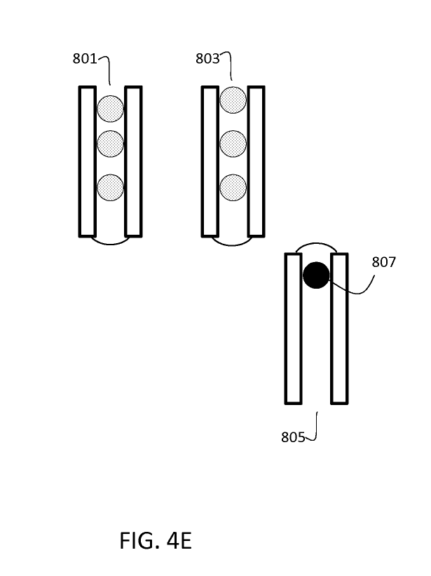

As shown in FIG. 4A, microfluidic channels 801 and 803 contain carrier fluid and at least one partition. Microfluidic channel 805 is not aligned with either microfluidic channel 801 or 803. The carrier fluid and the partitions are not able to flow out of the open end of microfluidic channels 801 or 803. FIG. 4B shows microfluidic channel 803 aligned with microfluidic channel 805, thereby allowing flow of carrier fluid and partitions between microfluidic channel 803 and microfluidic channel 805. FIG. 4C shows an embodiment in which microfluidic channel 805 received a partition when aligned to microfluidic channel 803, and the alignment is then disengaged. FIG. 4D shows microfluidic channel 805 aligned with microfluidic channel 801 to receive carrier fluid and a partition from microfluidic channel 801. The partitions within microfluidic channel 805 can placed close together, causing passive merging. Within microfluidic channel 805, the partitions are coalesced 807. As discussed above, coalesces may be accomplished by the application of an electric field. In partitions where cells are not present, electrodes may be located proximate to microfluidic channels to create an electric field to cause partitions to coalesce. In partitions in which cells are not present, passive merging can be accomplish by positioning partitions next to one another. In some embodiments in which partitions are present, aligning microfluidic channels to position partitions close together causes passive merging, as described above.

It should be appreciated that an open channel generally will include characteristics that facilitate control over fluid transport, e.g., structural characteristics (an elongated indentation) and/or physical or chemical characteristics (hydrophobicity vs. hydrophilicity) and/or other characteristics that can exert a force (e.g., a containing force) on a fluid. The fluid within the channel may partially or completely fill the channel. In some cases the fluid may be held or confined within the channel or a portion of the channel in some fashion, for example, using surface tension (e.g., such that the fluid is held within the channel within a meniscus, such as a concave or convex meniscus). In an article or substrate, some (or all) of the channels may be of a particular size. For example, channels may have a largest dimension perpendicular to fluid flow ranging from 5 mm to about 10 nm. Of course, in some cases, larger channels, tubes, etc. can be used to store fluids in bulk or deliver a fluid to the channel. In one embodiment, the channel is a capillary.

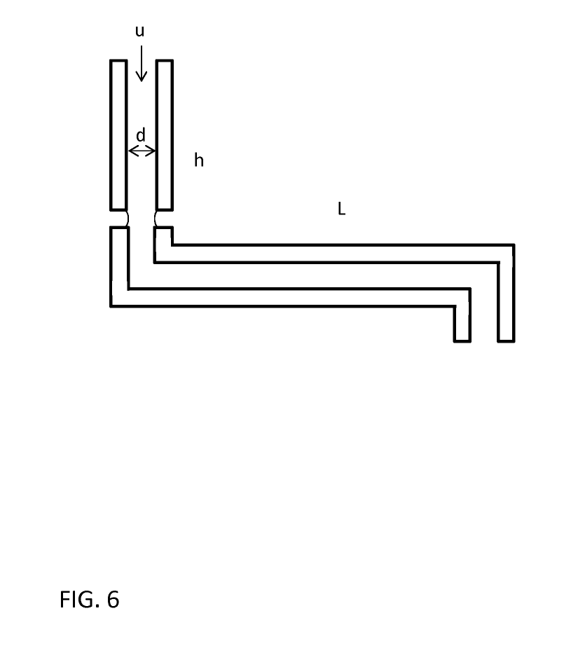

The microfluidic channels of the invention are configured such that liquid is retained within the microfluidic channel when it is completely out of alignment with another microfluidic channel (e.g., no overlap between open ends of channels). Liquid may be retained within the microfluidic channel due to forces such as surface tension. The flow in a microfluidic channel system, as shown in FIG. 6, with a height of h, an internal diameter of d, a length of L, a fluid velocity of u, a fluid density of ρ, gravitation force of g, fluid viscosity of μ, and surface tension of γ, can be represented by the equation:

$$2 ρ g h = \frac{γμ u ( 2 h + L )}{{( \frac{d}{2} )}^{2}}$$

or, rearranged as:

$$u = \frac{ ρ g {hd}^{2}}{2 γ μ ( 2 h + L )}$$

When fluid does not flow in the system, at maximum height, the equation becomes h=μγ/dρg.

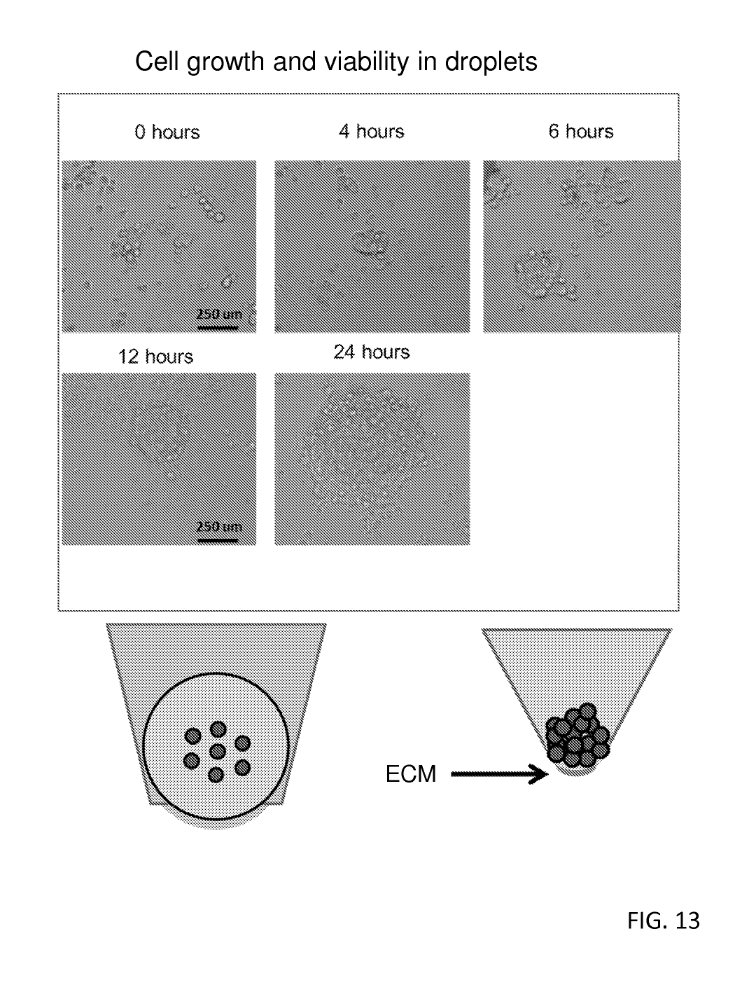

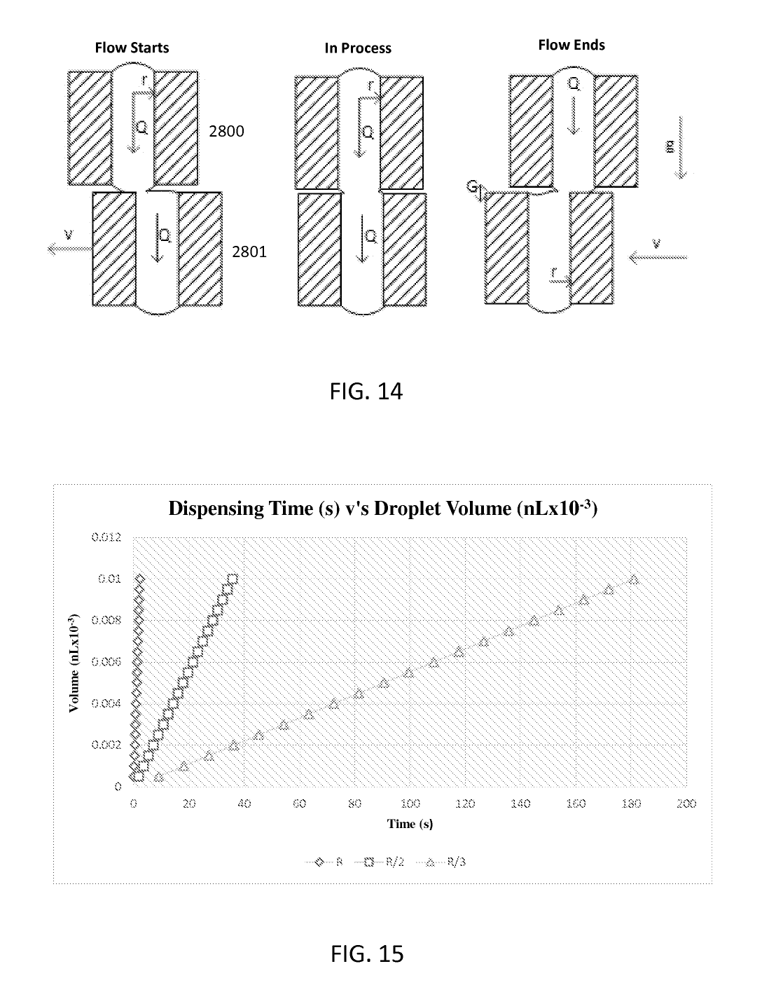

The volume of fluid that flows from one channel to another channel depends on the amount of time that the channels are aligned. As shown in FIG. 14, two channels 2800 and 2801 are aligned. Q is the flow rate in each channel, v is the velocity of the sliding channel, and r is the radius of the channel. Time when flowing is equal to nr/v, where n is the fraction of the lateral distance. As channel 2801 moves at a velocity relative to channel 2800, a volume of fluid flows from channel 2800 into channel 2801. G is the gap between the channels, and g is the force of gravity. The following equations denote the time required to dispense a volume, V from one channel to another channel. R is the resistance, P is the pressure, and u is the velocity.

R=(8 μL)/(πr̂4), ΔP=QR, ΔP=ρgh

∴ρgh=Q((8 μL)/(πr̂4))=πr̂2 u((8 μL)/(πr̂4))

∴ρgh=u((8 μL)/r̂2)

Qt=V where V=volume dispensed.

ρgh=V/t ((8 μL)/(πr̂4))

For a given volume displaced we look to minimise time t.

t=V/ρgh ((2 μL)/(ρr̂4))

∴t=(8 μLV)/(ρghπr̂4)

h=L for vertical channels

∴t=(8 μV)/(ρgπr̂4) This equation denotes the time required to dispense a volume, V.

FIG. 15 is a graph showing dispensing time (t) versus droplet volumes produced (nL) for varying vena contracta. The vena contracta means that average r is constantly changing, and can be averaged to r/2.

The dimensions of the channel may be chosen such that fluid is able to freely flow through the channel when channels are aligned and will not flow when channels are out of alignment with each other. The dimensions of the channel may also be chosen, for example, to allow a certain volumetric or linear flow rate of fluid in the channel. Of course, the number of channels and the shape of the channels can be varied by any method known to those of ordinary skill in the art.

The channels of the device of the present invention can be of any geometry as described. However, the channels of the device can comprise a specific geometry such that the contents of the channel are manipulated, e.g., sorted, mixed, prevent clogging, etc. For example, for channels that are configured to carry partitions, the channels of the device may preferably be square, with a diameter between about 2 microns and 1 mm. This geometry facilitates an orderly flow of partitions in the channels.

To prevent material (e.g., cells and other particles or molecules) from adhering to the sides of the channels, the channels (and coverslip, if used) may have a coating which minimizes adhesion. Such a coating may be intrinsic to the material from which the device is manufactured, or it may be applied after the structural aspects of the channels have been microfabricated. Any suitable material may be used for coating a surface of a channel. One such material is polytetrafluoroethylene (PTFE), sold under the trademark TEFLON by E.I. du Pont de Nemours and Company (Wilmington, Del.). The surface of the channels of the microfluidic system can be coated with any anti-wetting or blocking agent for the dispersed phase. The channel can be coated with any protein to prevent adhesion of the biological/chemical sample. For example, in one embodiment the channels are coated with BSA, PEG-silane and/or fluorosilane. For example, 5 mg/ml BSA is sufficient to prevent attachment and prevent clogging. In another embodiment, the channels can be coated with a cyclized transparent optical polymer obtained by copolymerization of perfluoro (alkenyl vinyl ethers), such as the type sold by Asahi Glass Co. under the trademark Cytop. In such an embodiment, the coating is applied from a 0.1-0.5 wt % solution of Cytop CTL-809M in CT-Solv 180. This solution can be injected into the channels of a microfluidic device via a plastic syringe. The device can then be heated to about 90° C. for 2 hours, followed by heating at 200° C. for an additional 2 hours. In another embodiment, the channels can be coated with a hydrophobic coating of the type sold by PPG Industries, Inc. under the trademark Aquapel (e.g., perfluoroalkylalkylsilane surface treatment of plastic and coated plastic substrate surfaces in conjunction with the use of a silica primer layer) and disclosed in U.S. Pat. No. 5,523,162, which patent is hereby incorporated by reference. By fluorinating the surfaces of the channels, the continuous phase preferentially wets the channels and allows for the stable generation and movement of partitions through the device. The low surface tension of the channel walls thereby minimizes the accumulation of channel clogging particulates. The surface of the channels in the microfluidic device can be also fluorinated to prevent undesired wetting behaviors. For example, a microfluidic device can be placed in a polycarbonate dessicator with an open bottle of (tridecafluoro-1,1,2,2-tetrahydrooctyl)trichlorosilane. The dessicator is evacuated for 5 minutes, and then sealed for 20-40 minutes. The dessicator is then backfilled with air and removed. This approach uses a simple diffusion mechanism to enable facile infiltration of channels of the microfluidic device with the fluorosilane and can be readily scaled up for simultaneous device fluorination.

Formation of microfluidic substrates are well known in the art. The substrates may be formed by several different types of materials, such as silicon, plastic, quartz, glass, plastic, or other suitable materials. Also, it should be appreciated that the size, shape and complexity of the microfluidic channels and structures that can be used in the microfluidic device depends on the materials used and the fabrication processes available for those materials. Typical system fabrication includes making trenches in a conducting material (silicon) or in a non-conducting substrate (e.g., glass or plastic) and converting them to channels by bonding a cover plate to the substrate. See for example, U.S. Pat. No. 6,210,986. In addition, for example, U.S. Pat. No. 5,885,470 teaches a microfluidic device having application in chemistry, biotechnology, and molecular biology that provides precise control of fluids by forming various grooves or channels and chambers in a polymeric substrate. The process of forming microfluidic channels in a substrate can include wet chemical etching, photolithographic techniques, controlled vapor deposition, and laser drilling into a substrate. Alternative techniques for constructing microfluidic channels may be employed in the fabrication of the device of the invention. For example, in Stjernstrom and Roeraade, Method for Fabrication of Microfluidic Systems in Glass, J. Micromechanics and Microenginneering, 8:33-38, 1998, walls are formed that define the channels rather than simply forming trenches in the substrate.

In some embodiments of the invention, a fluid within an open ended channel contains a partition, such as droplets, liposomes, emulsions, etc. The liquid partitions may have one compartment, or may have multiple compartments. The compartments may be aqueous based or oil based. A partition may contain both aqueous and oil based compartments. The compartment may contain cells, viruses, bacteria, molecules, compounds, elements, nucleic acids, etc., both in singular or plural form.

A fluid compartment may encase another fluid compartment. A fluid compartment may encase multiple fluid compartments. The fluid compartments within a fluid compartment may contain the same or different cell types. For example, a fluid compartment may contain a fluid compartment containing heart cells. Another fluid compartment may contain liver cells. Fluid compartments contained with a fluid compartment may contain the same or differing fluid types.

Methods of the invention may incorporate liposomes. A liposome is an artificially-prepared spherical vesicle composed of a lamellar phase lipid bilayer. Liposomes are often composed of phosphatidylcholine-enriched phospholipids and may also contain mixed lipid chains with surfactant properties such as egg phosphatidylethanolamine. A liposome design may employ surface ligands. A liposome encapsulates a region of aqueous solution inside a hydrophobic membrane. Hydrophobic chemicals can be dissolved into the membrane, and therefore, the liposome can carry both hydrophobic molecules and hydrophilic molecules. In some aspects of the invention, a fluid compartment may be a liposome, or a fluid compartment may contain liposomes.

Liposomes are prepared by disrupting biological membranes (such as by sonication). In one method of formation, liposomes are formed when thin lipid films or lipid cakes are hydrated and stacks of liquid crystalline bilayers become fluid and swell. The hydrated lipid sheets detach during agitation and self-close to form large, multilamellar vesicles (LMV) which prevents interaction of water with the hydrocarbon core of the bilayer at the edges. Once these particles have formed, reducing the size of the particle requires energy input in the form of sonic energy (sonication) or mechanical energy (extrusion).

Properties of lipid formulations can vary depending on the composition (cationic, anionic, neutral lipid species), however, similar preparation method can be used for all lipid vesicles regardless of composition. See for example “Liposomes in Gene Delivery,” Danilo D. Lasic, 1997, CRC Press LLC. Generally, the procedure involves preparation of the lipid for hydration, hydration with agitation, and sizing to a homogeneous distribution of vesicles. When preparing liposomes with mixed lipid composition, the lipids are dissolved and mixed in an organic solvent to assure a homogeneous mixture of lipids. Once the lipids are thoroughly mixed in the organic solvent, the solvent is removed to yield a lipid film. For small volumes of organic solvent (<1 mL), the solvent may be evaporated using a dry nitrogen or argon stream in a fume hood. For larger volumes, the organic solvent is removed by rotary evaporation yielding a thin lipid film on the sides of a round bottom flask. The lipid solution is transferred to containers and frozen by placing the containers on a block of dry ice or swirling the container in a dry ice-acetone or alcohol (ethanol or methanol) bath. Dry lipid films or cakes can be removed from the vacuum pump, the container close tightly and taped, and stored frozen until ready to hydrate. Hydration of the dry lipid film/cake is accomplished simply by adding an aqueous medium to the container of dry lipid and agitating. After addition of the hydrating medium, the lipid suspension should be maintained above the Tc during the hydration period. The hydration medium is generally determined by the application of the lipid vesicles. Suitable hydration media include distilled water, buffer solutions, saline, and nonelectrolytes such as sugar solutions. Physiological osmolality (290 mOsm/kg) is recommended for in vivo applications. Generally accepted solutions with meet these conditions are 0.9% saline, 5% dextrose, and 10% sucrose. During hydration some lipids form complexes unique to their structure. The product of hydration is a large, multilamellar vesicle (LMV). Once a stable, hydrated LMV suspension has been produced, the particles can be downsized by a variety of techniques, including sonication or extrusion. Disruption of LMV suspensions using sonic energy (sonication) typically produces small, unilamellar vesicles (SUV) with diameters in the range of 15-50 nm.

Second-generation liposomes may be incorporated into the present invention. Second-generation liposomes, or long-circulating liposomes, are obtained by modulating the lipid composition, size, and charge of the vesicle. The surface of the liposomes may be modified. For example, by incorporation of the synthetic polymer poly-(ethylene glycol) (PEG) in liposome composition. The presence of PEG on the surface of the liposomal carrier has been shown to extend blood-circulation time while reducing mononuclear phagocyte system uptake (stealth liposomes). See Immordino et al., Stealth liposomes: review of the basic science, rationale, and clinical applications, existing and potential, Int J Nanomedicine. September 2006; 1(3): 297-315.

Methods of the invention may incorporate micelles, an aggregate of surfactant molecules dispersed in a liquid colloid. A typical micelle in aqueous solution forms an aggregate with the hydrophilic “head” regions in contact with surrounding solvent, sequestering the hydrophobic single-tail regions in the micelle center. This phase is caused by the packing behavior of single-tail lipids in a bilayer. The difficulty filling all the volume of the interior of a bilayer, while accommodating the area per head group forced on the molecule by the hydration of the lipid head group, leads to the formation of the micelle. This type of micelle is known as a normal-phase micelle (oil-in-water micelle). Inverse micelles have the head groups at the center with the tails extending out (water-in-oil micelle). Micelles are approximately spherical in shape. Other phases, including shapes such as ellipsoids, cylinders, and bilayers, are also possible. The shape and size of a micelle are a function of the molecular geometry of its surfactant molecules and solution conditions such as surfactant concentration, temperature, pH, and ionic strength. The process of forming micelles is known as micellisation and forms part of the phase behavior of many lipids according to their polymorphism.

Methods of the invention may incorporate emulsions or droplets. An emulsion or droplet is an isolated portion of a first fluid that substantially or completely surrounded by a second fluid. In some cases, the first and second fluid may be completely surrounded by a third fluid.

The fluids may be different fluids, or two fluids may be the same. For example, the first fluid may be an aqueous fluid, the second fluid may be an oil, and the third fluid may be an aqueous fluid. In other cases, the first fluid may be an oil, the second fluid may be an aqueous fluid, and the third fluid may be an oil. For example, droplets may also include water-in-oil-in-water (water/oil/water) emulsions. These droplets are composed of three layers: an internal aqueous phase layer, an inner oil phase layer, and an external aqueous phase layer. The droplet may contain an external oil phase layer. In this embodiment, the external oil layer encompasses two aqueous compartments. In some embodiments the oil phase layer is a thin layer separating the internal and external aqueous phases. The thin oil phase layer has a high permeability, serving as a thin membrane between the two aqueous compartments. The oil phase layer can be less than 50 microns thick, less than 30 microns thick, less than 20 microns thick, less than 10 microns thick, less than 5 microns thick, or less than 1 micron thick. In another embodiment, the external oil layer contains multiple aqueous compartments, where each aqueous compartment is surrounded by an oil layer.

The droplets, whether containing two fluids or multiple fluids, may be spherical or substantially spherical; however, in other cases, the droplets may be non-spherical. For example, the droplets may have the appearance of blobs or other irregular shapes, for instance, depending on the external environment. As used herein, a first fluid is surrounded by a second fluid if a closed loop can be drawn or idealized around the first fluid through only the second fluid. An oil or an aqueous fluid phase can include a biological/chemical material. The biological/chemical material can be tissues, cells, particles, proteins, antibodies, amino acids, nucleic acids, nucleotides, small molecules, and pharmaceuticals. The biological/chemical material can include one or more labels known in the art. The label can be a DNA tag, dyes or quantum dot, or combinations thereof.

As used herein, the term emulsion generally refers to a preparation of one liquid distributed in small globules (also referred to herein as drops or droplets) in the body of a second liquid. The first and second fluids are immiscible with each other. For example, the discontinuous phase can be an aqueous solution and the continuous phase can be a hydrophobic fluid such as an oil. This is termed a water-in-oil emulsion. Alternatively, the emulsion may be an oil in water emulsion. In that example, the first liquid, which is dispersed in globules, is referred to as the discontinuous phase, whereas the second liquid is referred to as the continuous phase or the dispersion medium. The continuous phase can be an aqueous solution and the discontinuous phase is a hydrophobic fluid, such as an oil (e.g., decane, tetradecane, silicon, corn oil or hexadecane). The droplets or globules of oil in an oil in water emulsion are also referred to herein as “micelles”, whereas globules of water in a water in oil emulsion may be referred to as “reverse micelles”.

In the present application, the partitions may be liposomes, micelles, emulsions, droplets, etc. or any combination thereof. Also, a partition may include multiple compartments, or other partitions within a partition. For example, a liposome can contain at least one other liposome. A liposome may contain an emulsion, or a droplet. A liposome may contain multiple emulsions or droplets. A droplet may contain at least one liposome. It should be appreciated by one of skill in the art that the partitions can be arranged in any configuration.

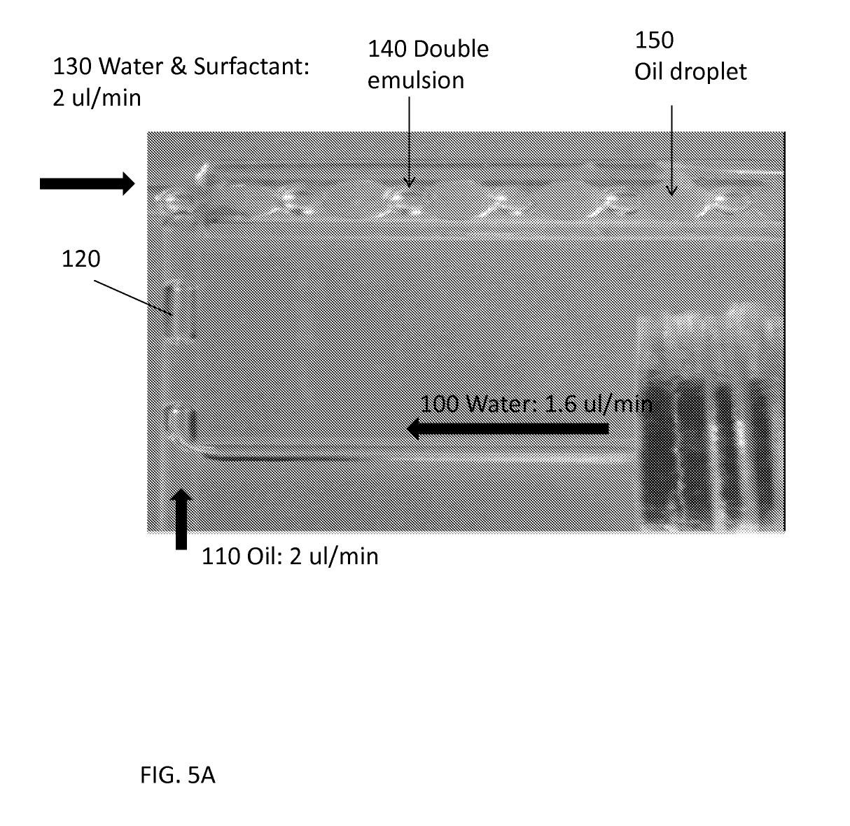

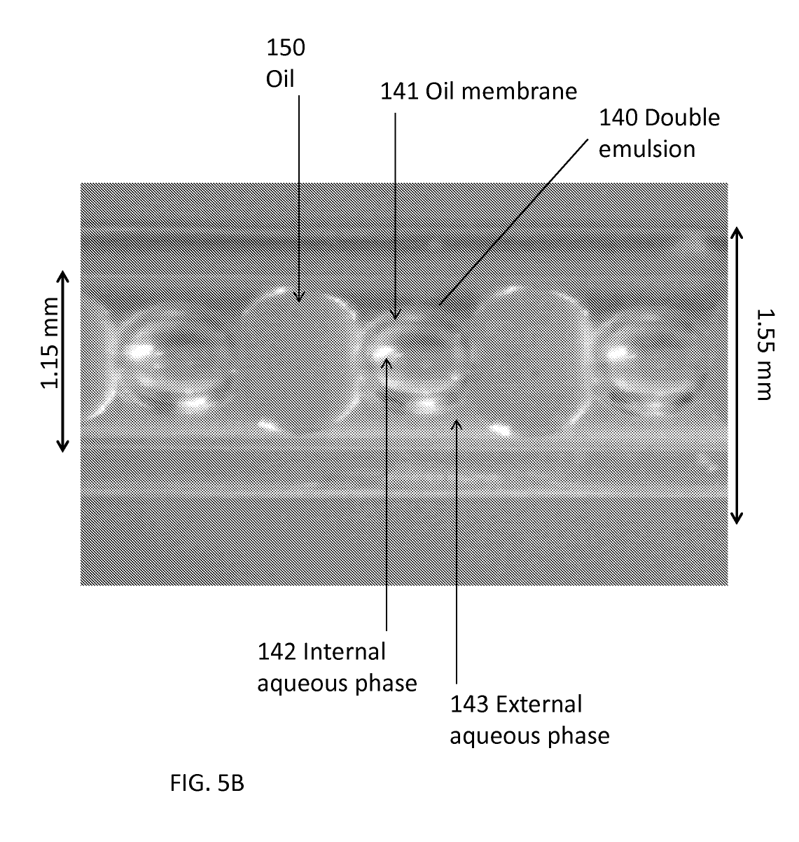

Partitions with multiple compartments may be formed by using channels and flowing streams of fluids. As shown in FIG. 5A, an aqueous fluid 100 is flowed in a channel. The fluid can be flowed at any speed or velocity, for example, at a rate of 0.1-1.0 microliters/min, at a rate of 1.0-2.0 microliters/min, or a rate greater than 2.0 microliters/min. The aqueous fluid 100 is flowed into an oil stream 110, or an immiscible fluid. The immiscible fluid 110 may be flowed at any rate, including at a rate of 0.1-1.0 microliters/min, at a rate of 1.0-2.0 microliters/min, or a rate greater than 2.0 microliters/min. The flowing of aqueous fluid 100 into an immiscible fluid creates aqueous partitions 120 in oil. An aqueous fluid containing a surfactant 130 is flowed into the stream of aqueous partitions 120 in oil. The rate of flow can be at a rate of 0.1-1.0 microliters/min, at a rate of 1.0-2.0 microliters/min, or a rate greater than 2.0 microliters/min. From the flowing of the aqueous fluid containing a surfactant 130 into the stream of aqueous fluids 120 in oil, double emulsions 140 and oil droplets 150 are formed.

As shown in FIG. 5B, the oil droplets 150 separate the double emulsions 140. The double emulsions 140 contain an oil membrane 141, an internal aqueous phase 142, and an external aqueous phase 143. Thus, methods of the invention may utilize partitions that have multiple compartments. For example, in FIG. 5B, a stem cell may be contained within the internal aqueous phase (142). The external aqueous phase 143 may contain a virus, a drug compound, or other agent that is able to transverse the oil membrane 141 and contact the stem cell. After a period of time, the double emulsion may be flowed into another microfluidic channel with a fresh external aqueous phase to impart a drug molecule to the external aqueous phase 143. The removal or addition of a fluid, such as removal and replacement of an external aqueous phase creates a concentration gradient with the internal aqueous phase, and causes diffusion across the oil membrane 141. The creation of a concentration gradient can remove or add contents to the internal aqueous phase 142.

It should be appreciated that these double emulsions may be referred to herein as partitions, and may be used in the microfluidic systems described herein to carry out the methods described herein.

In certain embodiments, the partitions are aqueous partitions that are surrounded by an immiscible carrier fluid. In other embodiments, the partitions are non-aqueous partitions surrounded by an immiscible fluid, such as oil partitions in a water continuous phase. In some embodiments, the sample fluid is aqueous, such as when employing a culture medium. The sample fluid is typically an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. Any liquid or buffer that is physiologically compatible with the sample can be used. In the preferred embodiment, the sample fluid comprises cell medium, as disclosed above. The sample fluid comprises the necessary components to ensure cell health and growth. As discussed above, any laboratory produced or commercially available cell medium may be employed.

As discussed above, the carrier fluid is one that is immiscible with the sample fluid. The carrier fluid can be a non-polar solvent, decane (e g., tetradecane or hexadecane), fluorocarbon oil, silicone oil or another oil (for example, mineral oil). In a preferred embodiment of the invention, the carrier fluid has a high surface tension and therefore, is retained a microfluidic channel at an open end. In an aspect of the invention, the carrier fluid forms a meniscus at the open end of the microfluidic channel, caused by surface tension. The meniscus can be either convex or concave, depending on the carrier fluid and the surface of the microfluidic channel. The partitions, whether including one or multiple partitions or compartments, may each be substantially the same shape and/or size. Alternatively, a first type partition may be considerably larger than a second type partition. The shape and/or size can be determined, for example, by measuring the average diameter or other characteristic dimension of the partitions. The average diameter of a plurality or series of partitions is the arithmetic average of the average diameters of each of the partitions. Those of ordinary skill in the art will be able to determine the average diameter (or other characteristic dimension) of a plurality or series of partitions, for example, using laser light scattering, microscopic examination, or other known techniques. The diameter of a partition, in a non-spherical partition, is the mathematically-defined average diameter of the partition, integrated across the entire surface. The average diameter of a partition (and/or of a plurality or series of partitions) may be, for example, less than 3 mm, less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers in some cases. The average diameter may also be at least about 1 micrometer, at least about 2 micrometers, at least about 3 micrometers, at least about 5 micrometers, at least about 10 micrometers, at least about 15 micrometers, or at least about 20 micrometers in certain cases. Partitions may vary in size, where a first type partition is larger than a second type partition.

In some embodiments, the aqueous phase is typically an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. Any liquid or buffer that is physiologically compatible with the population of molecules, cells or particles to be analyzed and/or sorted can be used. For example, in some embodiments, the aqueous fluid may be cell medium, for either maintaining or growing cells. The aqueous phase or aqueous liquid may be immiscible with the oil phase liquid, such as a non-polar solvent, decane (e g., tetradecane or hexadecane), fluorocarbon oil, corn oil, silicone oil or another oil (for example, mineral oil).

In certain embodiments, the carrier fluid contains one or more additives, such as agents which increase, reduce, or otherwise create non-Newtonian surface tensions (surfactants) and/or stabilize partitions against spontaneous coalescence on contact. Surfactants can include Tween, Span, fluorosurfactants, and other agents that are soluble in oil relative to water. In some applications, performance is improved by adding a second surfactant, or other agent, such as a polymer or other additive, to the sample fluid. Surfactants can aid in controlling or optimizing partition size, flow and uniformity. Furthermore, the surfactant can serve to stabilize aqueous emulsions in fluorinated oils from coalescing.

In certain embodiments, the partitions, or fluids within the partitions, may be coated with or contain a surfactant or a mixture of surfactants. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglycerl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates).

The compartments, whether aqueous or oil based, may contain a cell, a plurality of cells, or agents. Agents can include molecules, compounds, elements, drug molecules, ions, nucleic acids, etc. The compartments may contain any kind of cell, and compartments proximate to one another may contain differing or similar cells. The compartments may contain biological/chemical material (e.g., molecules, cells, or other particles) for combination, analysis and/or sorting in a microfluidic device. In some embodiments, the partitions may contain more than one particle or can contain no more than one particle. For example, where the biological material comprises cells, each partition may contain, on average, no more than one cell. However, in some embodiments, each partition may contain multiple cells. The partitions can be detected and/or sorted according to their contents.

The concentration (i.e., number) of molecules, compounds, viruses, bacteria, cells or particles in a partition can influence sorting efficiently and therefore is preferably optimized. In some embodiments, the concentration may be dilute enough that most of the partitions contain no more than a single molecule, cell or particle, with only a small statistical chance that a partition will contain two or more molecules, cells or particles. This may be to ensure that for the large majority of measurements, the level of reporter measured in each partition as it passes through the detection module corresponds to a single molecule, cell or particle and not to two or more molecules, cells or particles. In other embodiments, the partitions may contain a plurality or non-diluted concentration of cells, viruses, bacteria, molecules, compounds, etc.

The fluidic partition may contain additional entities, for example, other chemical, biochemical, or biological entities (e.g., dissolved or suspended in the fluid), cells, particles, gases, molecules, or the like. For example, a partition may contain cells and molecules, or cells and viruses. A partition does not need to be homogeneous. In some cases, the partition may each be substantially the same shape or size, as discussed above. In certain instances, the invention provides for the production of partition consisting essentially of a substantially uniform number of entities of a species therein (i.e., molecules, cells, particles, etc.). For example, about 90%, about 93%, about 95%, about 97%, about 98%, or about 99%, or more of a plurality or series of partition may each contain the same number of entities of a particular species. For instance, a substantial number of fluidic partition produced, e.g., as described above, may each contain 1 entity, 2 entities, 3 entities, 4 entities, 5 entities, 7 entities, 10 entities, 15 entities, 20 entities, 25 entities, 30 entities, 40 entities, 50 entities, 60 entities, 70 entities, 80 entities, 90 entities, 100 entities, etc., where the entities are molecules or macromolecules, cells, particles, etc., or any combination thereof. In some cases, the partition may each independently contain a range of entities, for example, less than 20 entities, less than 15 entities, less than 10 entities, less than 7 entities, less than 5 entities, or less than 3 entities in some cases. In some embodiments, a partition may contain 100,000,000 entities. In other embodiments, a partition may contain 1,000,000 entities.

In a liquid containing partition of fluid, some of which contain a species of interest and some of which do not contain the species of interest, the partition of fluid may be screened or sorted for those partition of fluid containing the species as further described below (e.g., using fluorescence or other techniques such as those described above), and in some cases, the partitions may be screened or sorted for those partition of fluid containing a particular number or range of entities of the species of interest, e.g., as previously described. Thus, in some cases, a plurality or series of fluidic partitions, some of which contain the species and some of which do not, may be enriched (or depleted) in the ratio of partition that do contain the species, for example, by a factor of at least about 2, at least about 3, at least about 5, at least about 10, at least about 15, at least about 20, at least about 50, at least about 100, at least about 125, at least about 150, at least about 200, at least about 250, at least about 500, at least about 750, at least about 1000, at least about 2000, or at least about 5000 or more in some cases. In other cases, the enrichment (or depletion) may be in a ratio of at least about 104, at least about 105, at least about 106, at least about 107, at least about 108, at least about 109, at least about 1010, at least about 1011, at least about 1012, at least about 1013, at least about 1014, at least about 1015, or more. For example, a fluidic partition containing a particular species may be selected from a library of fluidic partition containing various species, where the library may have about 100, about 103, about 104, about 105, about 106, about 107, about 108, about 109, about 1010, about 1011, about 1012, about 1013, about 1014, about 1015, or more items, for example, a DNA library, an RNA library, a protein library, a combinatorial chemistry library, etc. In certain embodiments, the partitions carrying the species may then be fused, reacted, or otherwise used or processed, etc., as further described below, for example, to initiate or determine a reaction.

In some aspects of the invention the partition may comprise sample fluid, discussed below. It should be appreciated that the sample fluid varies depending on the biological or chemical assay being performed within the droplet. In assays involving biological processes, the sample fluid is typically an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. In assays involving amplification and detection of nucleic acids, any liquid or buffer that is physiologically compatible with nucleic acid molecules can be used.

In assays related to cell culturing, the sample fluid, or any fluid within any fluid compartment, may comprise cell medium. The cell medium provides the necessary nutrients, growth factors, and hormones for cell growths, as well as regulating the pH and the osmotic pressure of the culture. The cell culture medium may allow for and support growth of the cells thus being cultured, or the cell medium is a maintenance medium. Growth is understood as an increase in viable cell density during at least a certain period of the cell culture. A maintenance medium is a cell culture medium which supports cell viability but which does not encourage cell growth. See for example, cell culture medium related patents: U.S. Pat. No. 4,038,139, 1977; U.S. Pat. No. 7,258,998, 2007; U.S. patent application Ser. No. 13/497,707, 2010; U.S. Pat. No. 8,338,177, 2012; and U.S. patent application Ser. No. 13/695,002, 2011. The growth medium controls the pH of the culture and buffers the cells in culture against fluctuations in the pH. This buffering may be achieved by including an organic (e.g., HEPES) or CO2 bicarbonate based buffer. Control of pH is needed to ensure the growth and health of cells in culture. Most normal mammalian cell lines grow well at pH 7.4, and there is very little variability among different cell strains.

Methods of the invention can incorporate any type of cell. In some embodiments, stem cells may be used. Induced pluripotent stem cells (also known as iPS cells or iPSCs) are a type of pluripotent stem cell that can be generated directly from adult cells. iPSCs are typically derived by introducing a specific set of pluripotency-associated genes, or “reprogramming factors,” into a given cell type. The original set of reprogramming factors (also dubbed Yamanaka factors) are the genes Oct4 (Pou5fl), Sox2, cMyc, and Klf4.

Somatic cells may be reprogrammed into induced pluripotent stem cells (iPSCs) using known methods such as the use of defined transcription factors. The iPSCs are characterized by their ability to proliferate indefinitely in culture while preserving their developmental potential to differentiate into derivatives of all three embryonic germ layers. In certain embodiments, fibroblasts are converted to iPSC by methods such as those discussed in Takahashi and Yamanaka, 2006, Induction of pluripotent stem cells from mouse embryonic and adult fibroblast cultures by defined factors Cell 126:663-676.; and Takahashi, et al., 2007, Induction of pluripotent stem cells from adult human fibroblasts by defined factors, Cell 131:861-872.

Induction of pluripotent stem cells from adult fibroblasts can be done by methods that include introducing four factors, Oct3/4, Sox2, c-Myc, and Klf4, under ES cell culture conditions. Human dermal fibroblasts (HDF) are obtained. A retroviruse containing human Oct3/4, Sox2, Klf4, and c-Myc is introduced into the HDF. Six days after transduction, the cells are harvested by trypsinization and plated onto mitomycin C-treated SNL feeder cells. See, e.g., McMahon and Bradley, 1990, Cell 62:1073-1085. About one day later, the medium (DMEM containing 10% FBS) is replaced with a primate ES cell culture medium supplemented with 4 ng/mL basic fibroblast growth factor (bFGF). See Takahashi, et al., 2007, Cell 131:861. Later, hES cell-like colonies are picked and mechanically disaggregated into small clumps without enzymatic digestion. Each cell should exhibit morphology similar to that of human ES cells, characterized by large nuclei and scant cytoplasm. The cells after transduction of HDF are human iPS cells. DNA fingerprinting, sequencing, or other such assays may be performed to verify that the iPS cell lines are genetically matched to the donor.

Methods of the present invention allow for iPS cells to be transfected to model a particular disease. iPS cells can also be used in modeling of infectious diseases. Human induced pluripotent stem cells (iPSCs) offer the ability to produce host-specific differentiated cells and thus have the potential to transform the study of infectious disease; and iPSC models of infectious disease have been described. Hepatocyte-like cells derived from iPSCs support the entire life cycle of hepatitis C virus, including inflammatory responses to infection, enabling studies of how host genetics impact viral pathogenesis. See Schwartz et al., 2012, PNAS 109(7):2544-2548.

The partitions may contain any biological or non-biological agent. In some embodiments, the partitions contain cells. In some embodiments, the partitions contain at least one molecule, at least one compound, or at least one element. In some embodiments, the partitions contain iPS cells. In some embodiments, the partitions contain iPS cells differentiated into varying cell types. A partition may contain one cell type while another partition contains a different cell type. For example, a partition may contain heart cells while another partition contains lung cells. The cells may be proximate to allow for cell-cell communication or signaling, or the cells may be separated to reduce signaling or other forms of cell-cell communication.

In certain embodiments, methods of the invention are used for chemical synthesis reactions or biological or chemical assays, such as sample preparation and analysis in a variety of fields in the art, including without limitation for many fields such as DNA sequencing, microarray sample preparation, genotyping, gene expression, biodefense, food monitoring, forensics, disease modeling, drug investigations, proteomics and cell biology. However, it should be appreciated that any material or species may be enveloped in a partition and processed in the device of the invention. For example, samples may be obtained from animals (including humans) and encompass fluids, solids, tissues, and gases. Biological samples include, without limitation, cells and any components thereof, blood products, such as plasma, serum and the like, proteins, peptides, amino acids, polynucleotide, lipids, carbohydrates, and any combinations thereof. The sample may include chemicals, organic or inorganic, used to interact with the interactive material. Environmental samples include environmental material such as surface matter, soil, water, crystals and industrial samples.

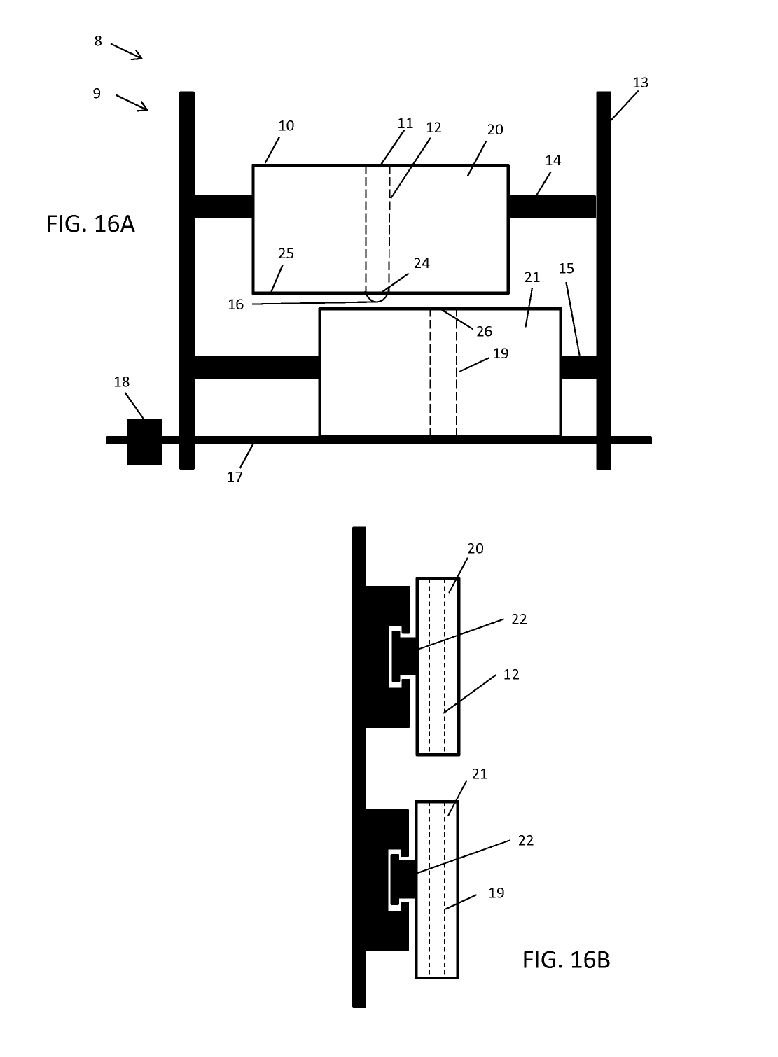

In some embodiments of the invention, as shown in FIGS. 16A and 16B, a microfluidic system 8 comprises a first substrate 20 with at least one reservoir 12 in fluid communication with an upper opening 11 at an upper surface 10 of the first substrate 20 and a lower opening 24 at a lower surface 25 of the first substrate 20, wherein the lower opening 24 is dimensioned such that a liquid 16 held in the reservoir 12 is prevented from flowing through the lower opening 24 and into ambient atmosphere by surface tension. The microfluidic system 8 also comprises a mechanical subsystem 9 supporting the first substrate 20 in an orientation such that liquid dispensed to the upper opening 11 flows into the reservoir 12. The mechanical subsystem 9 comprises an upper rail 14 and a lower rail 15. The microfluidic system 8 also comprises a second substrate 21 coupled to 22 and controlled by the mechanical subsystem 9, the second substrate 21 comprising a receptacle 19 open to a surface 26 of the second substrate 21, wherein operation of the mechanical subsystem 9 while a liquid 16 is held within the reservoir 12 brings the second substrate 21 into contact with a surface of the liquid 16, causing the liquid 16 to flow into the receptacle 19.

In some embodiments the systems are coupled or operably linked to a mechanical subsystem 9 comprising a chassis 13. A substrate of the invention may be operably coupled to a drive rail 17 which is operably connected to a motor 18, such that operation of the motor causes movement of the drive rail 17 which slides a substrate 21 which is coupled to a rail 15 by an attachment configuration 22.

Methods of the present invention may incorporate cell culturing. Cell culturing devices are commercially available, however, each of the currently available systems have at least, the following limitations: large size; high cost (particularly robotics); possible contamination, use of large scale cultures; treating cells with an enzyme; requiring complex software and using a large cell volume. Commercially available cell automation systems are developed by combining robotic stages, however, these systems are expensive given the cost of the robotic arms and the software used to drive them. There are a number of other automated cell systems commercially available, see for example the PANsys3000 system, which is a highly automated cell-culture system manufactured by Pan-SysTech; the CompacT SelecT system, which is an automated cell culture and assay-ready plating system manufactured by the Automation Partnership; Tecan, Inc. manufacturers several automated devices for cell culturing; Hamilton Robotics offers an extensive range of robotic systems for incorporation into cell culturing systems; and an automated cell culture system manufactured by MatriCal Bioscience.

The specific culture conditions may vary depending on the cell type. However, most culture conditions consist of a suitable vessel containing a substrate or medium that supplies the essential nutrients (amino acids, carbohydrates, vitamins, and minerals), growth factors, hormones and gases (O2, CO2). Furthermore, the physicochemical environment (pH, osmotic pressure, temperature) must be regulated. Cells may be grown floating in the culture medium (suspension culture) or grown while attached to a solid or semi-solid substrate (adherent or monolayer culture). In some embodiments, the devices and systems may be kept in an isothermal environment.

Cells are grown and maintained at an appropriate temperature and gas mixture (typically, 37° C., 5% CO2 for mammalian cells), usually in a cell incubator. Culture conditions vary widely for each cell type, and variation of conditions for a particular cell type can result in different phenotypes. See for example, Tsai et al., “Isolation of human multipotent mesenchymal stem cells from second-trimester amniotic fluid using a novel two-stage culture protocol,” Hum. Reprod. (2004) 19 (6): 1450-1456. doi: 10.1093/humrep/deh279, where amniotic fluid-derived mesenchymal stem cells (AFMSCs) were cultured to confluence and shifted to osteogenic medium (α-MEM supplemented with 10% FBS, 0.1 μmol/l dexamethason, 10 mmol/l β-glycerol phosphate, 50 μmol/l ascorbate) and adipogenic medium (α-MEM supplemented with 10% FBS, 1 μmol/l dexamethasone, 5 μg/ml insulin, 0.5 mmol/l isobutylmethylxanthine and 60 μmol/l indomethacin) for 3 weeks. For differentiation of neural cells, AFMSCs were incubated with α-MEM supplemented with 20% FBS, 1 mmol/l (3-mercaptoethanol, 5 ng/ml bFGF (Sigma, St Louis) for 24 h, and then treated with serum depletion for 5 h.

The cell medium provides the necessary nutrients, growth factors, and hormones for cell growths, as well as regulating the pH and the osmotic pressure of the culture. The cell culture medium according to the present invention is a medium allowing for and supporting growth of the animal cells thus cultured. Growth is understood as an increase in viable cell density during at least a certain period of the cell culture. According to the present invention, such definition of ‘growth medium’ is to be understood as being opposed to the term ‘maintenance medium’ in its usual meaning in the art. A maintenance medium is a cell culture medium which supports cell viability but which does not encourage cell growth. Often, such maintenance media do not contain essential growth factors such as transferrin, insulin, albumin and the like. See for example, cell culture medium related patents: U.S. Pat. No. 4,038,139; U.S. Pat. No. 7,258,998; and U.S. Pat. No. 8,338,177, each incorporated by reference.

The growth medium controls the pH of the culture and buffers the cells in culture against fluctuations in the pH. This buffering may be achieved by including an organic (e.g., HEPES) or CO2 bicarbonate based buffer. Control of pH is needed to ensure the growth and health of cells in culture. Most normal mammalian cell lines grow well at pH 7.4, and there is very little variability among different cell strains. However, some transformed cell lines have been shown to grow better at slightly more acidic environments (pH 7.0-7.4), and some normal fibroblast cell lines prefer slightly more basic environments (pH 7.4-7.7). Because the pH of the medium is dependent on the delicate balance of dissolved carbon dioxide (CO2) and bicarbonate (HCO3), changes in the atmospheric CO2 can alter the pH of the medium. Therefore, it is necessary to use exogenous CO2 when using media buffered with a CO2 bicarbonate based buffer, especially if the cells are cultured in open dishes or transformed cell lines are cultured at high concentrations. While most researchers usually use 5-7% CO2 in air, 4-10% CO2 is common for most cell culture experiments. However, each medium has a recommended CO2 tension and bicarbonate concentration to achieve the correct pH and osmolality.

The optimal temperature for cell culture largely depends on the body temperature of the host from which the cells were isolated, and to a lesser degree on the anatomical variation in temperature (e.g., temperature of the skin may be lower than the temperature of skeletal muscle). Overheating is a more serious problem than under heating for cell cultures; therefore, often the temperature in the incubator is set slightly lower than the optimal temperature. Most human and mammalian cell lines are maintained at 36° C. to 37° C. for optimal growth. Insect cells are cultured at 27° C. for optimal growth; they grow more slowly at lower temperatures and at temperatures between 27° C. and 30° C. Above 30° C., the viability of insect cells decreases, and the cells do not recover even after they are returned to 27° C. Avian cell lines require 38.5° C. for maximum growth. Although these cells can also be maintained at 37° C., they will grow more slowly. Cell lines derived from cold-blooded animals (e.g., amphibians, cold-water fish) tolerate a wide temperature range between 15° C. and 26° C. The consequences of deviating from the culture conditions required for a particular cell type can range from the expression of aberrant phenotypes to a complete failure of the cell culture.

Subculturing, or passaging, is the removal of the medium and transfer of cells from a previous culture into fresh growth medium, a procedure that enables the further propagation of the cell line or cell strain. Traditionally, to keep the cells at an optimal density for continued growth and to stimulate further proliferation, the culture has to divided and fresh medium supplied. For example, subculturing could be needed if a drop in pH is observed. A drop in the pH of the growth medium usually indicates a buildup of lactic acid, which is a by-product of cellular metabolism. Lactic acid can be toxic to the cells, and the decreased pH can be sub-optimal for cell growth.

As cells generally continue to divide in culture, they generally grow to fill the available area or volume. This can generate several issues: nutrient depletion in the growth media; changes in pH of the growth media; and accumulation of dead cells. Cell-to-cell contact can stimulate cell cycle arrest, causing cells to stop dividing, known as contact inhibition. Cell-to-cell contact can also stimulate cellular differentiation. Genetic and epigenetic alterations, with a natural selection of the altered cells potentially leading to overgrowth of abnormal, culture-adapted cells with decreased differentiation and increased proliferative capacity. Therefore, processing of the cells to ensure removal of harmful species and replenishment of cell medium is needed every 1-3 days, depending on the particular protocol.

Traditionally, cell viability is determined by staining the cells with trypan blue. As trypan blue dye is permeable to non-viable cells or death cells whereas it is impermeable to this dye. Stain the cells with trypan dye and load to haemocytometer and calculate % of viable cells. Cell viability is calculated as the number of viable cells divided by the total number of cells within the grids on the hemacytometer. If cells take up trypan blue, they are considered non-viable. It would be appreciated by one skilled in the art for the optical detection of cells containing trypan blue.

In the present invention, cells are encapsulated in partitions containing cell medium. The cells can be flowed from various chambers via the multichannel system of the invention. Microfluidic channels can be aligned to cause flow of the partitions into a humidifier chamber. For example, mammalian cells are grown in humidified atmosphere at 37° C. and 5% CO2, in cell culture incubators. Microfluidic channels can be aligned to deliver CO2 to the humidifier chamber within the multichannel system by aligning microfluidic channels that seal, or by an inlet microfluidic channel configured to deliver liquids or gases to the chamber. It should be appreciated that alignment of microfluidic channels of the invention can be sealed to allow for the transport of gases. To replenish or re-suspend the cells in fresh growth medium, which could be required every 2-3 days, the partitions are flowed from a chamber into the microfluidic channels to be merged or coalesced with fresh growth medium. It should be appreciated that partitions containing cells during culturing may be retained within microfluidic channels or chambers of the present invention. After merging or coalesced with fresh growth medium, the partitions may be retained within microfluidic channels, or the cells may be diverted to a chamber.

In an embodiment of the invention, cells are grown in nanoliter-microliter partitions in cell medium that is replenished every 2-3 days. In some assays, cells may require splitting every 2-3 days. Media change involves adding one or more partitions of fresh media to a partition of incubated cells and thereby partially replenishing growth media. Merging of partitions is discussed above. Cells are further incubated in the combined partition or in smaller partitions generated by splitting the combined partition. Cell subculture or splitting is achieved similarly to media change by combining (merging and mixing) a partition of incubated cells and a partition of fresh media, splitting the combined partition, and repeating this procedure using the split partition(s) until a desired cell concentration is reached. Final partitions are then incubated, while other partitions of suspended cells generated in the subculturing process are discarded. Incubation can be accomplished within the microfluidic channels of the device, or in chambers of the device. One or more thermal regulators can be employed to ensure proper temperature.

In a multiplexed assay, multiple partitions containing one kind or multiple kinds of cells are exposed to partitions containing one or multiple reagents and are assayed similarly to the assays described above. A multiplex device can also be used for multiplex cell culture, where cells can be grown and maintained in multiple partitions.

Employing the methods discussed herein, iPS cells are encased in partitions containing cell medium and related growth factors. The iPS cell partitions are cultured by techniques and methods discussed above, or known in the art. iPS cell partitions can be merged with other materials and reactants, including maintenance medium, transfection reagents, etc. during the incubation process.

iPS cell partitions may be used in expression profiling where target compounds are introduced into the partitions by methods disclosed herein. In order to use expression analysis for disorder diagnosis, a threshold of expression is established. The threshold may be established by reference to literature or by using a reference sample from a subject known not to be afflicted with the disorder. The expression may be over-expression compared to the reference (i.e., an amount greater than the reference) or under-expression compared to the reference (i.e., an amount less than the reference). In expression profiling, the iPS cell partitions are merged with partitions containing target compounds and allowed to further incubate, which may involve splitting of partitions, i.e. splitting of cell cluster, and merging of partitions, i.e. introduction of freshcell medium. Methods of the invention may be used to detect any disorder or compound effect. The iPS cell partitions may be flowed passed a detector to screen for abnormalities, or diverted to a collection chamber for analysis.

In some embodiments, genetically modified cells are used in the methods of the invention. In some embodiments, genetic editing of stem cells or transfected stem cells is employed. Genetic or genome editing techniques may proceed by any suitable method such as zinc-finger domain methods, transcription activator-like effector nucleases (TALENs), or clustered regularly interspaced short palindromic repeat (CRISPR) nucleases. Genome editing may be used for, e.g., knocking out a gene, introducing a premature stop codon, interfering with a promoter region, or changing the function of an ion channel or other cellular protein. In certain embodiments, genome editing techniques are applied to the iPS cells. Using genome editing for modifying a chromosomal sequence, a control cell or cell line can be generated, or any other genetic variant of the first cell may be created.

### EXAMPLE 1

Using the methods and systems of the invention described above, cells are maintained within droplets and monitored over a twenty-four hour period.

MCF7 breast cancer cells are contained within 30 μL droplets. Using the system described above, at least one breast cancer cell is contained within a droplet. The droplets containing the MCF7 breast cancer cells are exposed to a solution containing a dissolved drug molecule. This can be accomplished by merging a droplet containing a breast cancer cell with a droplet containing a drug molecule. Additionally, instead of containing just one drug molecule, the solution may contain multiple drug molecules to study the effects of drug combinations on the cells, such as the MCF7 breast cancer cell. Alternatively, the droplets containing the breast cancer cells are loading into a channel of the present invention. A shuttle aligns with a channel containing a droplet containing a cancer cell. The shuttle aligns for a period of time so only one droplet flows from the channel into the shuttle. The shuttle slides to align with another channel containing a solution. The shuttle aligns for a period of time to allow fluid from the channel to flow into the shuttle. The fluid containing the breast cancer cell and the solution containing the drug solution merge to form a droplet. It should be appreciated that the shuttle speed and alignment time controls the volume of the fluid flowing from the channel into the shuttle. Also, the shuttle can align to allow any combination of the individual solutions to flow into the shuttle, thereby exposing the cells to any combination of the drug molecules. The droplets containing the breast cancer cells and the drug molecules are incubated within the system of the invention.

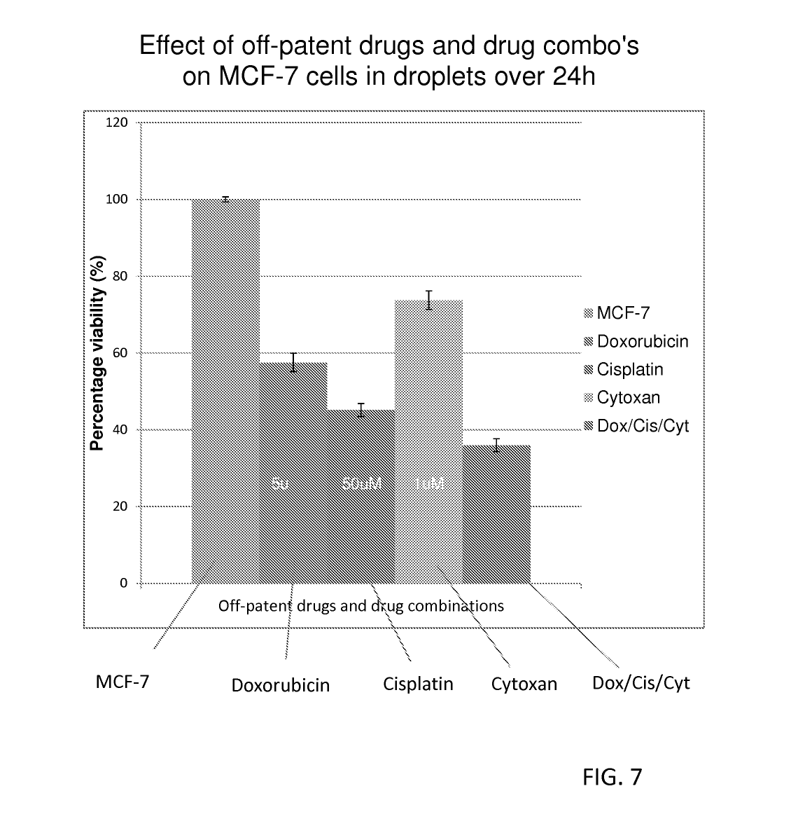

Using the methods and devices of the invention, a plurality of droplets of approximately 30 μL in volume containing at least one breast cancer cell and a fluid volume are formed. The fluid may comprise a control solution (lacking a drug molecule), may comprise at least one type of drug molecule, or may comprise multiple drug molecules. Droplets containing the MCF7 breast cancer cell and the drug molecule Doxorubicin are formed. Droplets containing the MCF7 breast cancer cell and the drug molecule Cisplatin are formed. Droplets containing the MCF7 breast cancer cell and the drug molecule Cytoxan are formed. Droplets containing the MCF7 breast cancer cell and the drug molecules Doxorubicin, Cisplatin, and Cytoxan are formed. After formation, the droplets dwell within the systems of the invention for 24 hours. After 24 hours, the droplets are analyzed for cell viability. The droplets are counted to determine cell viability. FIG. 7 reports the viability of the cells within the droplets over a 24 hour period. As shown in FIG. 7, the droplets containing the MCF7 breast cancer cells shows a nearly 100% viability after 24 hours. Droplets containing the MCF7 breast cancer cells and the drug Doxoruicin yield 58% viability after 24 hours. Droplets containing the MCF7 breast cancer cells and the drug Cisplatin showed 45% cell viability after 24 hours. Droplets containing the MCF7 breast cancer cells and the drug Cytoxan showed 73% cell viability after 24 hours. Droplets containing the MCF7 breast cancer cells and the drug combination of Doxorubicin, Cisplatin, and Cytoxan showed 34% cell viability after 24 hours. From this experiment, the control saw a nearly 100% cell viability while the MCF7 breast cancer cells exposed to at least one drug saw a reduction in cell viability. Therefore, using the methods and systems of the invention, cells can be maintained within the system, exposed to various drug combinations, and analyzed to determine the effects of the drug or drug combination on the cells. It should be appreciated that other cell characteristics can be measured to determine the effects of the drug molecules on the cells. For example, protein levels can be analyzed, enzyme levels can be analyzed, or the cells can be assayed for the presence or absence of a cell product or cellular component.

### EXAMPLE 2

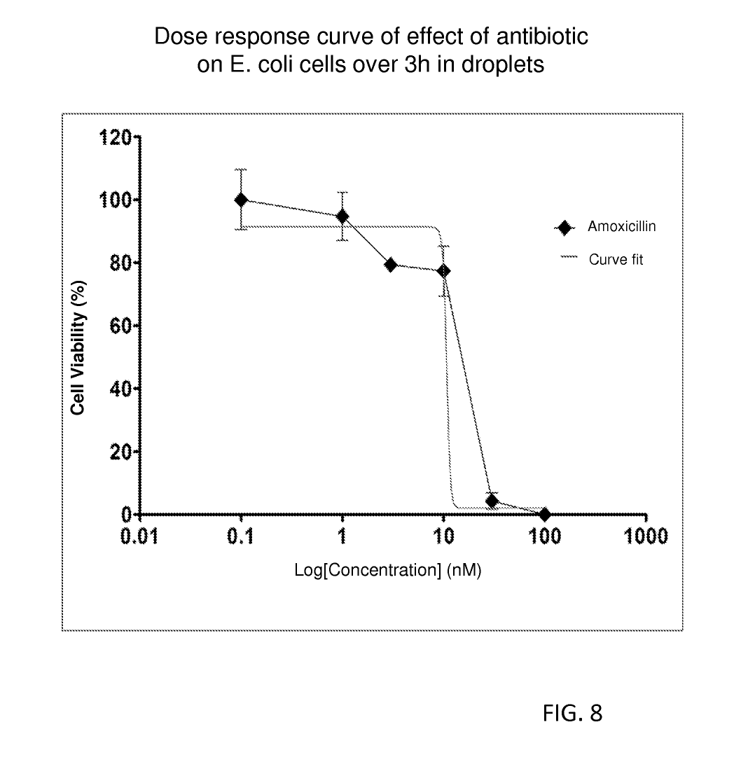

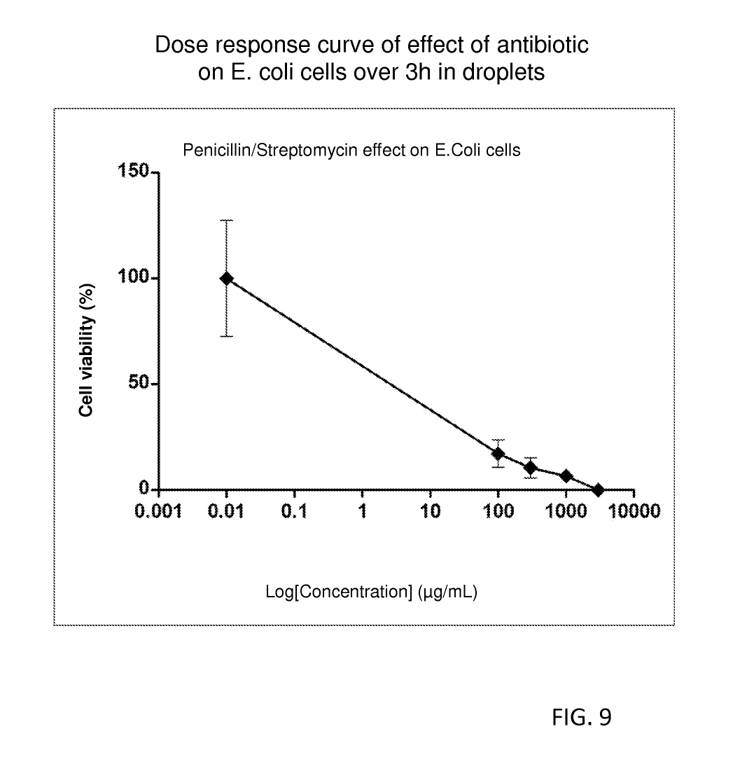

The methods and systems of the invention can be used to determine dose response curves. In this example, droplets containing at least one bacterial cell of E. coli were monitored over a three hour period. The droplet containing the E.coli cells were exposed to various concentrations of the antibiotic Amoxicillin. After three hours, the droplets were analyzed for cell viability. As shown in FIG. 8, cell viability was reduced over increasing values in Amoxicillin concentration. Using the systems and methods of the invention, concentration of drugs that causes cell death can be determined. As shown in FIG. 9, dose response curves were developed for the determination of the combination of Penicillin and Streptomycin on E. Coli cells. The antibiotics Penicillin and Streptomycin were contained within 30 μL droplets containing E. Coli cells. The cells were analyzed after three hours for cell viability. The results are reported in FIG. 9. Using the systems and methods of the invention, concentrations of a drug that result in cell death can be determined. It should be appreciated that the cells may be assayed by any known technique in the art. For example, the cells may be analyzed for the expression of a protein, or for the absence of a protein. The cells may be analyzed for any change in cellular function or for any cellular product.

### EXAMPLE 3

Using the systems and methods of the present invention, effects of drug molecules on cells are investigated.

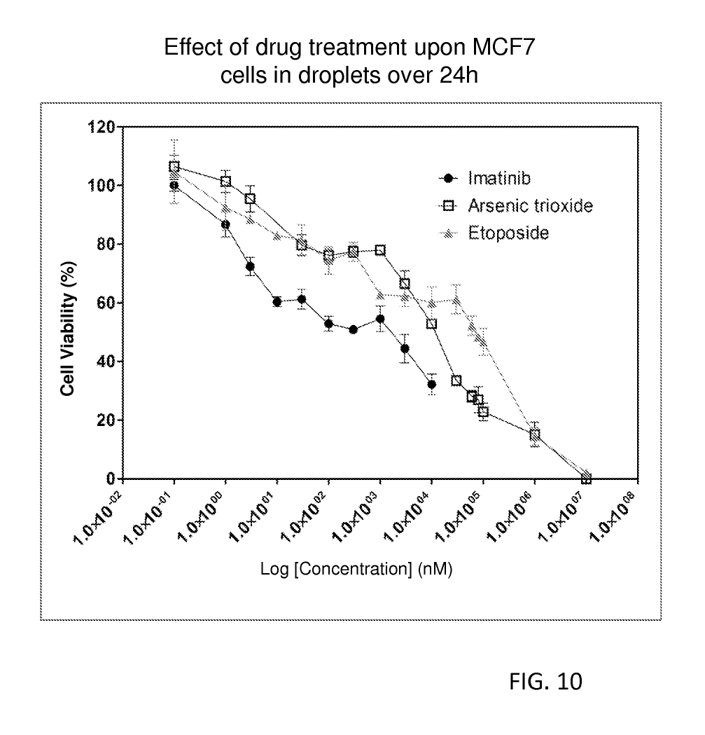

MCF-7 breast cancer cells are contained within 30 μL droplets. It should be appreciated that droplets of any volume can be created by the system of the invention. A plurality of droplets containing MCF-7 cells was created using the systems of the invention. MCF-7 cell containing droplets were exposed to the drug Imatinib. A plurality of droplets was exposed to the drug at various concentrations. MCF-7 cell containing droplets were exposed to the drug arsenic trioxide. A plurality of droplets was exposed to the drug at various concentrations. MCF-7 cell containing droplets were exposed to the drug etoposide. A plurality of droplets was exposed to the drug at various concentrations. The cells were cultured in the system of the invention and then analyzed after a 24 hour period. The plurality of droplets was analyzed for cell viability. FIG. 10 shows the dose response curve for each of the drugs at the various concentrations. As shown in FIG. 10, the optimum concentration for the drug to kill the cancer cells can be determined.

### EXAMPLE 4

Using the systems and methods of the present invention, effects of drug molecules on iPSC are investigated.

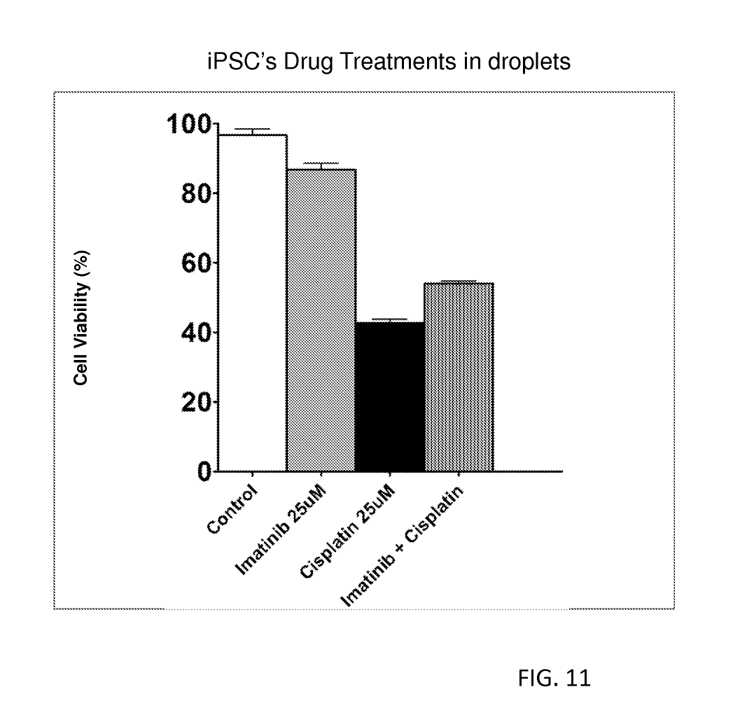

iPSC were contained within droplets using the systems and methods of the invention. The cells were cultured in 1 μL droplets and monitored over a 24 hour period. Some of the droplets were exposed to Imatinib, Cisplatin, and the combination of Imatinib and Cisplatin. As shown in FIG. 11, the combination of Imatinib and Cisplatin showed an increase in cell viability compared to Cisplatin alone. Thus, using systems and methods of the invention, possible synergistic effects of drug molecules can be probed and investigated.

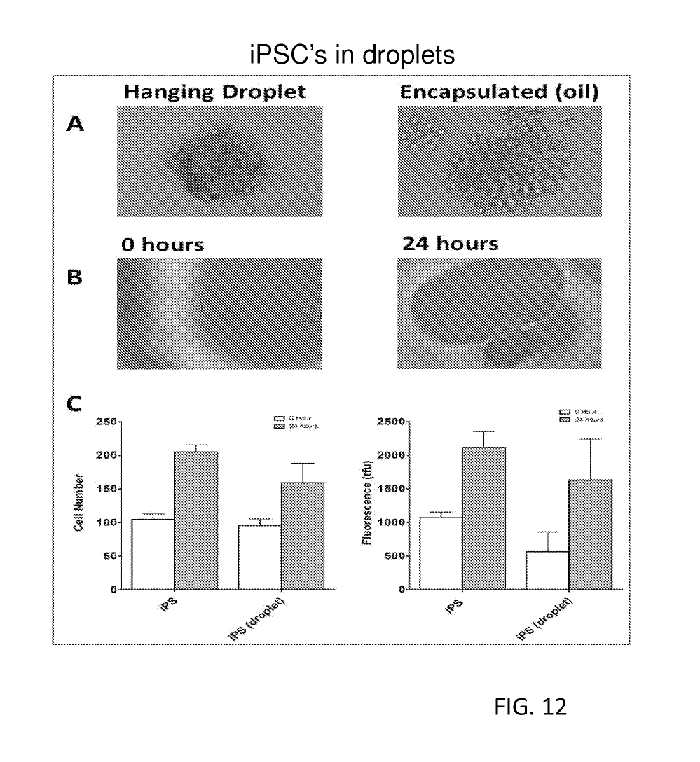

FIGS. 12, 13A and 13B show the results of culturing and growing cells in droplets formed and maintained in the systems of the invention. In panel A of FIG. 12, iPSCs were cultured in 20 μL hanging droplets in open-ended well plates, encapsulated in oil. See co-pending application 62/115,877 filed Feb. 13, 2015, the contents of which is incorporated by reference in its entirety. The iPS cells shown in panel B of FIG. 12 were cultured in 1 μL hanging droplets in open-ended well plates. As shown in panel C of FIG. 12, the cell number increased, or the collection of cells grew over a 24 hour period in both 20 μL hanging droplets and 1 μL hanging droplets. Similarly, FIG. 13 shows that the number of cells increase over a 24 hour period.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for analyzing cells in partitions, the method comprising:
containing at least one cell in a fluid partition in a first open-ended channel;
introducing a fluid comprising an agent into the partition by aligning at least a portion of the first open ended channel with a second open ended channel from which the fluid is provided; and
analyzing the contents of the fluid partition.

2. The method of claim 1, wherein aligning comprises contacting a liquid in the first channel with a liquid in the second channel to cause fluid to flow.

3. The method of claim 1, wherein the at least one cell is a stem cell.

4. The method of claim 1, wherein the at least one cell is a pluripotent stem cell.

5. The method of claim 1, wherein the at least one cell is a differentiated pluripotent stem cell.

6. The method of claim 4, wherein the pluripotent stem cell models a disease state.

7. The method of claim 3, wherein the stem cell is infected with a contagious disease.

8. The method of claim 3, wherein the stem cell is exposed to a virus, pathogen, or bacteria.

9. The method of claim 4, wherein the pluripotent stem cell is exposed to an agent found within the fluid.

10. The method of claim 5, wherein a concentration gradient is created to remove waste from the fluid partition.

11. The method of claim 1, wherein the fluid contains at least one drug molecule.

12. The method of claim 1, wherein the method is performed in an isothermal environment.

13. A method for analyzing cells in partitions, the method comprising:
containing at least one cell in a first fluid compartment of a fluid partition in a first open ended channel, wherein the fluid partition comprises at least two fluid compartments;
introducing a fluid into the partition by aligning the first open ended channel with a second open ended channel from which the fluid is provided; and
analyzing the contents of the fluid partition.

14. The method of claim 13, wherein the first fluid compartment contains a first cell type and a second fluid compartment contains a second cell type.

15. The method of claim 14, wherein the first and second cell types are different.

16. The method of claim 14, wherein the first and second cell are the same.

17. The method of claim 13, wherein the first fluid compartment contains a stem cell.

18. The method of claim 17, wherein a second fluid compartment contains an agent.

19. The method of claim 18, wherein the agent is selected from the group consisting of a virus, a drug molecule, a bacterium, and a protein.

20. The method of claim 19, wherein the stem cell is analyzed after incubation with the agent.

21. The method of claim 13, wherein the method is conducted in an isothermal environment.
